(19) United States
(12) Patent Application Publication
(10) Pub. No.: US 2025/0206710 A1
(43) Pub. Date: Jun. 26, 2025

(54) COMPOSITIONS AND METHODS RELATED TO THE SIMULTANEOUS MANUFACTURE AND PURIFICATION OF FLAVOR AND FRAGRANCE MOLECULES

(71) Applicant: Natural Extraction Systems, LLC, Boulder, CO (US)

(72) Inventor: C. Russell Thomas, Boulder, CO (US)

(21) Appl. No.: 18/847,568

(22) PCT Filed: Mar. 15, 2023
(86) PCT No.: PCT/US23/64423

§ 371 Date: Sep. 16, 2024

Related U.S. Application Data
(60) Provisional application No. 63/320,058, filed on Mar. 15, 2022.

(51) Int. Cl. C07D 307/50; A23L 2/56; C07C 41/34; C11B 9/00

(52) U.S. Cl. CPC C07D 307/50; A23L 2/56; C07C 41/34; C11B 9/0061; C11B 9/0076

(57) ABSTRACT

This disclosure relates to the discovery that improved flavors and fragrances can be manufactured during a rapid heating and cooling process that performs simultaneous thermal decomposition of a reactant and distillation of the thermal decomposition product. Various aspects of this disclosure relate to the discovery that the rate of thermal decomposition and distillation can be varied to produce a range of flavor and fragrance molecules in formats compatible with direct addition to consumer products.

### BACKGROUND

The global flavors and fragrance industry is mature, and new flavors and fragrances are rare. The cost-effective production of innovative flavors and fragrances is desirable.

### SUMMARY

Various aspects of this disclosure relate to the discovery that improved flavors and fragrances can be manufactured during a rapid heating and cooling process that performs simultaneous thermal decomposition of a reactant and distillation of the thermal decomposition product. Various aspects of this disclosure relate to the discovery that the rate of thermal decomposition and distillation can be varied to produce a range of flavor and fragrance molecules in formats compatible with direct addition to consumer products. Wood extracts manufactured according to the methods of this disclosure can be used, for example, to enhance the aging of wine and spirits and to balance the flavor of beer. Wines produced from such extracts have been selected as markedly superior to untreated wines by trained sommeliers in double-blind tests. Nonalcoholic beverages produced from such extracts taste more like real alcoholic beverages compared to nonalcoholic beverages produced with conventional extracts. Conventional barrel aging and conventional wood extracts impart undesirable compounds into products such as tannins, which cause unfavorable astringency associated with dry-mouth and pucker. The methods of this disclosure favor the production of favorable flavors and fragrances while disfavoring the extraction of unfavorable compounds including tannins. Essential oils and downstream products produced by the methods of this disclosure are therefore differentiable as superior to essential oils and downstream products produced by existing methods. Additional features differentiate the aspects of this disclosure from existing compositions and methods as set forth in the detailed description and claims that follow, and this summary paragraph does not limit the disclosure or any patent claim that matures from this document.

### DETAILED DESCRIPTION

Various aspects of this disclosure relate to a method to manufacture furfural, comprising providing a composition comprising a pentose; transferring energy to the composition to convert the composition into a vapor and residual solids and liquids, wherein converting the composition into the vapor and the residual solids and liquids comprises converting a portion of the pentose into furfural vapor and wherein the vapor comprises the furfural vapor; separating the vapor from a majority of the residual solids and liquids; and condensing a portion of the vapor to produce a distillate that comprises furfural.

“Furfural” refers to furan-2-carbaldehyde.

“Comprising” refers to an open set; a method that comprises “separating the vapor from a majority of the residual solids and liquids”, for example, can also include separating the vapor from an additional portion of the residual solids and liquids, such as by first separating the vapor from the majority of the residual solids and liquids using a cyclone and then separating the vapor from the additional portion of the residual solids and liquids using a filter.

Various aspects of this disclosure relate to a method to manufacture 5-(hydroxymethyl) furfural, comprising providing a composition comprising a pentose; transferring energy to the composition to convert the composition into a vapor and residual solids and liquids, wherein converting the composition into the vapor and the residual solids and liquids comprises converting a portion of the pentose into 5-(hydroxymethyl)furfural vapor and wherein the vapor comprises the 5-(hydroxymethyl)furfural vapor; separating the vapor from a majority of the residual solids and liquids; and condensing a portion of the vapor to produce a distillate that comprises 5-(hydroxymethyl)furfural.

Various aspects of this disclosure relate to a method to manufacture 5-(methyl)furfural, comprising providing a composition comprising a pentose; transferring energy to the composition to convert the composition into a vapor and residual solids and liquids, wherein converting the composition into the vapor and the residual solids and liquids comprises converting a portion of the pentose into 5-(methyl)furfural vapor and wherein the vapor comprises the 5-(methyl)furfural vapor; separating the vapor from a majority of the residual solids and liquids; and condensing a portion of the vapor to produce a distillate that comprises 5-(methyl)furfural.

In some embodiments, the pentose has the chemical formula C₅H₁₀O₅. In some specific embodiments, the pentose is an aldehyde. In some very specific embodiments, the pentose is xylose. In some specific embodiments, the pentose is a ketone. In some very specific embodiments, the pentose is xylulose.

Various aspects of this disclosure relate to a method to manufacture guaiacol, comprising providing a composition comprising phenolic molecules selected from monolignols, lignans, and lignin; transferring energy to the composition to convert the composition into a vapor and residual solids and liquids, wherein converting the composition into the vapor and the residual solids and liquids comprises converting a portion of the phenolic molecules into guaiacol vapor and wherein the vapor comprises the guaiacol vapor; separating the vapor from a majority of the residual solids and liquids; and condensing a portion of the vapor to produce a distillate that comprises guaiacol.

“Guaiacol” refers to 2-methoxyphenol.

Various aspects of this disclosure relate to a method to manufacture eugenol, comprising providing a composition comprising phenolic molecules selected from monolignols, lignans, and lignin; transferring energy to the composition to convert the composition into a vapor and residual solids and liquids, wherein converting the composition into the vapor and the residual solids and liquids comprises converting a portion of the phenolic molecules into eugenol vapor and wherein the vapor comprises the eugenol vapor; separating the vapor from a majority of the residual solids and liquids; and condensing a portion of the vapor to produce a distillate that comprises eugenol.

“Eugenol” refers to 2-methoxy-4-(prop-2-enyl)phenol.

Various aspects of this disclosure relate to a method to manufacture vanillin, comprising providing a composition comprising phenolic molecules selected from monolignols, lignans, and lignin; transferring energy to the composition to convert the composition into a vapor and residual solids and liquids, wherein converting the composition into the vapor and the residual solids and liquids comprises converting a portion of the phenolic molecules into vanillin vapor and wherein the vapor comprises the vanillin vapor; separating the vapor from a majority of the residual solids and liquids; and condensing a portion of the vapor to produce a distillate that comprises vanillin.

“Vanillin” refers to 4-hydroxy-3-methoxybenzaldehyde.

Various aspects of this disclosure relate to a method to manufacture 2-methoxy-3-methylphenol, comprising providing a composition comprising phenolic molecules selected from monolignols, lignans, and lignin; transferring energy to the composition to convert the composition into a vapor and residual solids and liquids, wherein converting the composition into the vapor and the residual solids and liquids comprises converting a portion of the phenolic molecules into 2-methoxy-3-methylphenol vapor and wherein the vapor comprises the 2-methoxy-3-methylphenol vapor; separating the vapor from a majority of the residual solids and liquids; and condensing a portion of the vapor to produce a distillate that comprises 2-methoxy-3-methylphenol.

Various aspects of this disclosure relate to a method to manufacture 2-methoxy-4-methylphenol, comprising providing a composition comprising phenolic molecules selected from monolignols, lignans, and lignin; transferring energy to the composition to convert the composition into a vapor and residual solids and liquids, wherein converting the composition into the vapor and the residual solids and liquids comprises converting a portion of the phenolic molecules into 2-methoxy-4-methylphenol vapor and wherein the vapor comprises the 2-methoxy-4-methylphenol vapor; separating the vapor from a majority of the residual solids and liquids; and condensing a portion of the vapor to produce a distillate that comprises 2-methoxy-4-methylphenol.

Various aspects of this disclosure relate to a method to manufacture 2-methoxy-5-methylphenol, comprising providing a composition comprising phenolic molecules selected from monolignols, lignans, and lignin; transferring energy to the composition to convert the composition into a vapor and residual solids and liquids, wherein converting the composition into the vapor and the residual solids and liquids comprises converting a portion of the phenolic molecules into 2-methoxy-5-methylphenol vapor and wherein the vapor comprises the 2-methoxy-5-methylphenol vapor; separating the vapor from a majority of the residual solids and liquids; and condensing a portion of the vapor to produce a distillate that comprises 2-methoxy-5-methylphenol.

Various aspects of this disclosure relate to a method to manufacture syringol, comprising providing a composition comprising phenolic molecules selected from monolignols, lignans, and lignin; transferring energy to the composition to convert the composition into a vapor and residual solids and liquids, wherein converting the composition into the vapor and the residual solids and liquids comprises converting a portion of the phenolic molecules into syringol vapor and wherein the vapor comprises the syringol vapor; separating the vapor from a majority of the residual solids and liquids; and condensing a portion of the vapor to produce a distillate that comprises syringol.

“Syringol” refers to 2,6-dimethoxyphenol.

Various aspects of this disclosure relate to a method to manufacture syringaldehyde, comprising providing a composition comprising phenolic molecules selected from monolignols, lignans, and lignin; transferring energy to the composition to convert the composition into a vapor and residual solids and liquids, wherein converting the composition into the vapor and the residual solids and liquids comprises converting a portion of the phenolic molecules into syringaldehyde vapor and wherein the vapor comprises the syringaldehyde vapor; separating the vapor from a majority of the residual solids and liquids; and condensing a portion of the vapor to produce a distillate that comprises syringaldehyde.

“Syringaldehyde” refers to 4-hydroxy-3,5-dimethoxybenzaldehyde.

Various aspects of this disclosure relate to a method to manufacture 2,6-dimethoxy-3-methylphenol, comprising providing a composition comprising phenolic molecules selected from monolignols, lignans, and lignin; transferring energy to the composition to convert the composition into a vapor and residual solids and liquids, wherein converting the composition into the vapor and the residual solids and liquids comprises converting a portion of the phenolic molecules into 2,6-dimethoxy-3-methylphenol vapor and wherein the vapor comprises the 2,6-dimethoxy-3-methylphenol vapor; separating the vapor from a majority of the residual solids and liquids; and condensing a portion of the vapor to produce a distillate that comprises 2,6-dimethoxy-3-methylphenol.

Various aspects of this disclosure relate to a method to manufacture 2,6-dimethoxy-4-methylphenol, comprising providing a composition comprising phenolic molecules selected from monolignols, lignans, and lignin; transferring energy to the composition to convert the composition into a vapor and residual solids and liquids, wherein converting the composition into the vapor and the residual solids and liquids comprises converting a portion of the phenolic molecules into 2,6-dimethoxy-4-methylphenol vapor and wherein the vapor comprises the 2,6-dimethoxy-4-methylphenol vapor; separating the vapor from a majority of the residual solids and liquids; and condensing a portion of the vapor to produce a distillate that comprises 2,6-dimethoxy-4-methylphenol.

Various aspects of this disclosure relate to a method to manufacture 2,6-dimethylphenol, comprising providing a composition comprising phenolic molecules selected from monolignols, lignans, and lignin; transferring energy to the composition to convert the composition into a vapor and residual solids and liquids, wherein converting the composition into the vapor and the residual solids and liquids comprises converting a portion of the phenolic molecules into 2,6-dimethylphenol vapor and wherein the vapor comprises the 2,6-dimethylphenol vapor; separating the vapor from a majority of the residual solids and liquids; and condensing a portion of the vapor to produce a distillate that comprises 2,6-dimethylphenol.

Various aspects of this disclosure relate to a method to manufacture 2,6-dimethoxy-4-(prop-2-enyl)phenol, comprising providing a composition comprising phenolic molecules selected from monolignols, lignans, and lignin; transferring energy to the composition to convert the composition into a vapor and residual solids and liquids, wherein converting the composition into the vapor and the residual solids and liquids comprises converting a portion of the phenolic molecules into 2,6-dimethoxy-4-(prop-2-enyl)phenol vapor and wherein the vapor comprises the 2,6-dimethoxy-4-(prop-2-enyl)phenol vapor; separating the vapor from a majority of the residual solids and liquids; and condensing a portion of the vapor to produce a distillate that comprises 2,6-dimethoxy-4-(prop-2-enyl)phenol.

In some embodiments, the phenolic molecules comprise one, two, or each of para-coumaryl alcohol, coniferyl alcohol, and sinapyl alcohol.

“Para-coumaryl alcohol” refers to 4-(3-hydroxyprop-1-enyl)phenol.

“Coniferyl alcohol” refers to 4-(3-hydroxyprop-1-enyl)-2-methoxyphenol.

“Sinapyl alcohol” refers to 4-(3-hydroxyprop-1-enyl)-2,6-dimethoxyphenol.

In some embodiments, the phenolic molecules comprise lignans.

In some embodiments, the phenolic molecules comprise lignin.

In some embodiments, the composition comprises a pentose; converting the composition into the vapor and the residual solids and liquids comprises converting a portion of the pentose into furfural vapor; the vapor comprises the furfural vapor; and the distillate comprises furfural.

In some embodiments, the composition comprises a pentose; converting the composition into the vapor and the residual solids and liquids comprises converting a portion of the pentose into 5-(hydroxymethyl)furfural vapor; the vapor comprises the 5-(hydroxymethyl)furfural vapor; and the distillate comprises 5-(hydroxymethyl)furfural.

In some embodiments, the composition comprises a pentose; converting the composition into the vapor and the residual solids and liquids comprises converting a portion of the pentose into 5-(methyl)furfural vapor; the vapor comprises the 5-(methyl)furfural vapor; and the distillate comprises 5-(methyl)furfural.

In some embodiments, the pentose has the chemical formula C₅H₁₀O₅. In some specific embodiments, the pentose is an aldehyde. In some very specific embodiments, the pentose is xylose. In some specific embodiments, the pentose is a ketone. In some very specific embodiments, the pentose is xylulose.

In some embodiments, the composition comprises phenolic molecules selected from monolignols, lignans, and lignin; converting the composition into the vapor and the residual solids and liquids comprises converting a portion of the phenolic molecules into guaiacol vapor; the vapor comprises the guaiacol vapor; and the distillate comprises guaiacol.

In some embodiments, the composition comprises phenolic molecules selected from monolignols, lignans, and lignin; converting the composition into the vapor and the residual solids and liquids comprises converting a portion of the phenolic molecules into eugenol vapor; the vapor comprises the eugenol vapor; and the distillate comprises eugenol.

In some embodiments, the composition comprises phenolic molecules selected from monolignols, lignans, and lignin; converting the composition into the vapor and the residual solids and liquids comprises converting a portion of the phenolic molecules into vanillin vapor; the vapor comprises the vanillin vapor; and the distillate comprises vanillin.

In some embodiments, the composition comprises phenolic molecules selected from monolignols, lignans, and lignin; converting the composition into the vapor and the residual solids and liquids comprises converting a portion of the phenolic molecules into 2-methoxy-3-methylphenol vapor; the vapor comprises the 2-methoxy-3-methylphenol vapor; and the distillate comprises 2-methoxy-3-methylphenol.

In some embodiments, the composition comprises phenolic molecules selected from monolignols, lignans, and lignin; converting the composition into the vapor and the residual solids and liquids comprises converting a portion of the phenolic molecules into 2-methoxy-4-methylphenol vapor; the vapor comprises the 2-methoxy-4-methylphenol vapor; and the distillate comprises 2-methoxy-4-methylphenol.

In some embodiments, the composition comprises phenolic molecules selected from monolignols, lignans, and lignin; converting the composition into the vapor and the residual solids and liquids comprises converting a portion of the phenolic molecules into 2-methoxy-5-methylphenol vapor; the vapor comprises the 2-methoxy-5-methylphenol vapor; and the distillate comprises 2-methoxy-5-methylphenol.

In some embodiments, the composition comprises phenolic molecules selected from monolignols, lignans, and lignin; converting the composition into the vapor and the residual solids and liquids comprises converting a portion of the phenolic molecules into syringol vapor; the vapor comprises the syringol vapor; and the distillate comprises syringol.

In some embodiments, the composition comprises phenolic molecules selected from monolignols, lignans, and lignin; converting the composition into the vapor and the residual solids and liquids comprises converting a portion of the phenolic molecules into syringaldehyde vapor; the vapor comprises the syringaldehyde vapor; and the distillate comprises syringaldehyde.

In some embodiments, the composition comprises phenolic molecules selected from monolignols, lignans, and lignin; converting the composition into the vapor and the residual solids and liquids comprises converting a portion of the phenolic molecules into 2,6-dimethoxy-3-methylphenol vapor; the vapor comprises the 2,6-dimethoxy-3-methylphenol vapor; and the distillate comprises 2,6-dimethoxy-3-methylphenol.

In some embodiments, the composition comprises phenolic molecules selected from monolignols, lignans, and lignin; converting the composition into the vapor and the residual solids and liquids comprises converting a portion of the phenolic molecules into 2,6-dimethoxy-4-methylphenol vapor; the vapor comprises the 2,6-dimethoxy-4-methylphenol vapor; and the distillate comprises 2,6-dimethoxy-4-methylphenol.

In some embodiments, the composition comprises phenolic molecules selected from monolignols, lignans, and lignin; converting the composition into the vapor and the residual solids and liquids comprises converting a portion of the phenolic molecules into 2,6-dimethylphenol vapor; the vapor comprises the 2,6-dimethylphenol vapor; and the distillate comprises 2,6-dimethylphenol.

In some embodiments, the composition comprises phenolic molecules selected from monolignols, lignans, and lignin; converting the composition into the vapor and the residual solids and liquids comprises converting a portion of the phenolic molecules into 2,6-dimethoxy-4-(prop-2-enyl)phenol vapor; the vapor comprises the 2,6-dimethoxy-4-(prop-2-enyl)phenol vapor; and the distillate comprises 2,6-dimethoxy-4-(prop-2-enyl)phenol.

In some embodiments, the phenolic molecules comprise one, two, or each of para-coumaryl alcohol, coniferyl alcohol, and sinapyl alcohol.

In some embodiments, the phenolic molecules comprise lignans.

In some embodiments, the phenolic molecules comprise lignin.

In some embodiments, the composition is in fluid communication with a gas phase that has a temperature and a pressure; the composition off-gasses a molecule into the gas phase at a vaporization rate; a vaporized molecule recondenses from the gas phase onto the composition at a condensation rate; the molecule and the vaporized molecule have the same chemical structure; and the method comprises converting the molecule into the vaporized molecule at a mass transfer rate, which is equal to the vaporization rate minus the condensation rate.

In some embodiments, the molecule and the vaporized molecule are co-selected from furfural; 5-(hydroxymethyl)furfural; 5-(methyl)furfural; guaiacol; eugenol; vanillin; 2-methoxy-3-methylphenol; 2-methoxy-4-methylphenol; 2-methoxy-5-methylphenol; syringol; syringaldehyde; 2,6-dimethoxy-3-methylphenol; 2,6-dimethoxy-4-methylphenol; 2,6-dimethylphenol; and 2,6-dimethoxy-4-(prop-2-enyl)phenol, such that the molecule and the vaporized molecule are the same.

In some embodiments, the molecule and the vaporized molecule are both furfural.

In some embodiments, the molecule and the vaporized molecule are both 5-(hydroxymethyl)furfural.

In some embodiments, the molecule and the vaporized molecule are both 5-(methyl)furfural.

In some embodiments, the molecule and the vaporized molecule are both guaiacol.

In some embodiments, the molecule and the vaporized molecule are both eugenol.

In some embodiments, the molecule and the vaporized molecule are both vanillin.

In some embodiments, the molecule and the vaporized molecule are both 2-methoxy-3-methylphenol.

In some embodiments, the molecule and the vaporized molecule are both 2-methoxy-4-methylphenol.

In some embodiments, the molecule and the vaporized molecule are both 2-methoxy-5-methylphenol.

In some embodiments, the molecule and the vaporized molecule are both syringol.

In some embodiments, the molecule and the vaporized molecule are both syringaldehyde.

In some embodiments, the molecule and the vaporized molecule are both 2,6-dimethoxy-3-methylphenol.

In some embodiments, the molecule and the vaporized molecule are both 2,6-dimethoxy-4-methylphenol.

In some embodiments, the molecule and the vaporized molecule are both 2,6-dimethylphenol.

In some embodiments, the molecule and the vaporized molecule are both 2,6-dimethoxy-4-(prop-2-enyl)phenol.

In some embodiments, the molecule and the vaporized molecule are both water.

In some embodiments, transferring the energy comprises transferring sufficient energy to pyrolyze a portion of the composition.

In some embodiments, the method comprises bombarding the composition with at least 10 sextillion molecules of a sweep gas per gram of the composition. In some specific embodiments, the method comprises bombarding the composition with at least 10 sextillion molecules of a sweep gas per gram of the composition per second.

In some embodiments, the method comprises bombarding the composition with at least 1 liter of a sweep gas per gram of the composition. In some embodiments, the method comprises bombarding the composition with at least 1 liter of a sweep gas per gram of the composition per second.

In some embodiments, the method comprises bombarding the composition with a sweep gas at a force of at least 10 millinewtons per gram of the composition.

In some embodiments, the method comprises bombarding the composition with a sweep gas with an impulse of at least 10 millinewton-seconds per gram of the composition.

In some embodiments, the method comprises bombarding the composition with a sweep gas that has a kinetic energy of at least 1 millijoule per gram of the composition.

In some embodiments, the method comprises bombarding the composition with a sweep gas that has a velocity of at least 100 millimeters per second.

In some embodiments, the method comprises bombarding the composition with a sweep gas for no greater than 60 seconds.

In some embodiments, the method comprises bombarding the composition with a sweep gas for no greater than 240 seconds.

In some embodiments, the method comprises bombarding the composition with a sweep gas for at least 100 milliseconds and no greater than 10 seconds.

In some embodiments, the bombarding increases the mass transfer rate.

In some embodiments, the bombarding decreases the condensation rate.

In some embodiments, the bombarding increases the vaporization rate.

In some embodiments, the bombarding increases the vaporization rate to at least 5 micrograms of the molecule per gram of the composition per second.

In some embodiments, the bombarding increases the mass transfer rate to at least 5 micrograms of the molecule per gram of the composition per second.

In some embodiments, the vaporized molecule has a partial pressure at the surface of the composition; and the bombarding decreases the partial pressure of the vaporized molecule at the surface of the composition.

In some embodiments, the vaporized molecule has a partial pressure at the surface of the composition; the vaporized molecule recondenses onto the composition at a condensation rate; decreasing the partial pressure of the vaporized molecule at the surface of the composition decreases the condensation rate; and the bombarding both decreases the partial pressure of the vaporized molecule at the surface of the composition and decreases the condensation rate.

In some embodiments, the vaporized molecule has a partial pressure at the surface of the composition; decreasing the partial pressure of the vaporized molecule at the surface of the composition increases the mass transfer rate; and the bombarding both decreases the partial pressure of the vaporized molecule at the surface of the composition and increases the mass transfer rate.

In some embodiments, the vaporized molecule has a partial pressure in the gas phase; the partial pressure of the vaporized molecule has concentration gradients in the gas phase; the concentration gradients have magnitudes; and the bombarding decreases the magnitudes of the concentration gradients.

In some embodiments, the vaporized molecule has a partial pressure in the gas phase; the partial pressure of the vaporized molecule in the gas phase inversely correlates with distance from the composition; and the bombarding decreases the inverse correlation.

In some embodiments, the vaporized molecule has a partial pressure in the gas phase; the partial pressure of the vaporized molecule in the gas phase inversely correlates with distance from the composition; the inverse correlation has a magnitude; and the bombarding decreases the magnitude of the inverse correlation.

In some embodiments, the vaporized molecule has a partial pressure in the gas phase; the partial pressure of the vaporized molecule in the gas phase inversely correlates with distance from the composition; the inverse correlation has a correlation coefficient of at least −1 and less than 0, wherein −1 is complete inverse correlation and 0 is no correlation; the correlation coefficient has an absolute value; and the bombarding decreases the absolute value of the correlation coefficient.

In some embodiments, the bombarding performs work on the vaporized molecule. In some specific embodiments, the bombarding performs work on the vaporized molecule that translates the vaporized molecule in three-dimensional space. In some very specific embodiments, the bombarding performs work on the vaporized molecule that translates the vaporized molecule by at least 1 meter.

In some embodiments, the bombarding transfers kinetic energy to the vaporized molecule. In some specific embodiments, the bombarding transfers at least 10 microjoules of kinetic energy to the vaporized molecule per gram of the vaporized molecule.

In some embodiments, the bombarding accelerates the vaporized molecule. In some specific embodiments, the bombarding accelerates the vaporized molecule to an average velocity of at least 100 millimeters per second.

In some embodiments, the molecule has a vapor pressure; and the bombarding increases the vapor pressure of the molecule. In some specific embodiments, the molecule has a vapor pressure; increasing the vapor pressure of the molecule increases the vaporization rate; and the bombarding both increases the vapor pressure of the molecule and increases the vaporization rate. In some very specific embodiments, the bombarding increases the vaporization rate to at least 5 micrograms of the molecule per gram of the composition per second.

In some embodiments, the molecule has a vapor pressure; increasing the vapor pressure of the molecule increases the mass transfer rate; and the bombarding both increases the vapor pressure of the molecule and increases the mass transfer rate. In some specific embodiments, the bombarding increases the mass transfer rate to at least 5 micrograms of the molecule per gram of the composition per second.

In some embodiments, the molecule has a vapor pressure; the composition has thermal energy; the bombarding increases the thermal energy of the composition; and increasing the thermal energy of the composition increases the vapor pressure of the molecule.

In some embodiments, increasing the vapor pressure of the molecule comprises sensible heat transfer from the gas phase to the composition; the sensible heat transfer has a rate; and the bombarding increases the rate of the sensible heat transfer. In some specific embodiments, the sensible heat transfer from the gas phase to the composition is completed in less than 60 seconds.

In some embodiments, converting the molecule into the vaporized molecule comprises latent heat transfer between the composition and the gas phase; the latent heat transfer has a rate; and the bombarding increases the rate of the latent heat transfer. In some specific embodiments, the latent heat transfer between the composition and the gas phase is completed in less than 60 seconds.

In some embodiments, the bombarding suspends at least 75 percent of the composition in the gas phase. In some specific embodiments, the bombarding suspends at least 98 percent of the composition in the gas phase.

In some embodiments, suspension in the gas phase includes pneumatically entraining the composition in the gas phase such that the composition is translated from one location to another location. In some embodiments, suspending the composition in the gas phase includes fluidizing the composition in the gas phase in a fluidic bed.

In some embodiments, the bombarding occurs by a method in which the composition is remains stationary in a basket or cartridge. In some embodiments, the bombarding occurs by a method in which the composition is remains stationary on a surface or in a chamber.

In some embodiments, the bombarding performs work on the composition. In some specific embodiments, the bombarding performs work on the composition that translates at least 90 percent of the composition. In some very specific embodiments, the bombarding performs work on the composition that translates at least 90 percent of the composition by at least 1 meter.

In some embodiments, the bombarding transfers kinetic energy to the composition. In some specific embodiments, the bombarding transfers at least 10 microjoules of kinetic energy to the composition per gram of the composition.

In some embodiments, the bombarding accelerates the composition. In some specific embodiments, the bombarding accelerates at least 90 percent of the composition to an average velocity that is greater than 100 millimeters per second.

In some embodiments, the method comprises sensible heat transfer from the gas phase to the composition, wherein the sensible heat transfer has a rate; and the bombarding increases the rate of the sensible heat transfer.

In some embodiments, the composition has a temperature that is less than the temperature of the gas phase when the composition is provided; the method comprises heating the composition; and the bombarding heats the composition.

In some embodiments, the composition has a temperature of no greater than 100 degrees Celsius when the composition is provided; the method comprises heating the composition to a temperature greater than 100 degrees Celsius; and the bombarding heats the composition.

In some embodiments, the composition has a temperature of at least 15 degrees Celsius and no greater than 100 degrees Celsius when the composition is provided; the method comprises heating the composition to a temperature greater than 100 degrees Celsius; and the bombarding heats the composition.

In some embodiments, the bombarding performs work that separates the vaporized molecule from the majority of the residual solids and liquids.

In some embodiments, the bombarding propels the vaporized molecule through a cyclone or centrifugal separator that separates the vaporized molecule from the majority of the residual solids and liquids.

In some embodiments, the bombarding propels the composition through a cyclone or centrifugal separator that separates the vaporized molecule from the majority of the residual solids and liquids.

In some embodiments, the bombarding propels the vaporized molecule through a filter that separates the vaporized molecule from the majority of the residual solids and liquids.

In some embodiments, the method comprises providing a system comprising a first chamber and a second chamber, wherein converting the composition into the vapor and the residual solids and liquids is performed in the first chamber; the first chamber and the second chamber are in fluid communication such that the portion of the vapor can be propelled from the first chamber into the second chamber; the method comprises propelling the portion of the vapor from the first chamber to the second chamber; the bombarding propels the portion of the vapor from the first chamber to the second chamber; and condensing the portion of the vapor is performed in a second chamber.

In some embodiments, the bombarding propels the vaporized molecule to a heat sink that condenses the vaporized molecule.

In some embodiments, the sweep gas comprises one or more of molecular nitrogen, molecular oxygen, carbon dioxide, argon, neon, water vapor, and ethanol vapor. In some specific embodiments, the sweep gas comprises one or more of molecular nitrogen, molecular oxygen, carbon dioxide, argon, neon, water vapor, and ethanol vapor at a combined concentration of at least 50 percent by mass. In some very specific embodiments, the sweep gas consists of one or more of molecular nitrogen, molecular oxygen, carbon dioxide, argon, neon, water vapor, and ethanol vapor.

“Consists” refers to a closed set such that a sweep gas that consists of one or more of molecular nitrogen, molecular oxygen, carbon dioxide, argon, neon, water vapor, and ethanol vapor cannot also comprise, for example, methane.

In some embodiments, the sweep gas comprises molecular nitrogen. In some specific embodiments, the sweep gas comprises molecular nitrogen at a concentration of at least 50 percent by mass. In some very specific embodiments, the sweep gas consists of molecular nitrogen.

In some embodiments, the sweep gas is not wet steam.

In some embodiments, the sweep gas is not steam.

In some embodiments, the sweep gas lacks water vapor at a concentration greater than 90 percent by mass. In some specific embodiments, the sweep gas lacks water vapor at a concentration greater than 10 percent by mass.

In some embodiments, the sweep gas comprises dry steam. In some specific embodiments, the sweep gas comprises dry steam at a concentration of at least 50 percent by mass. In some very specific embodiments, the sweep gas consists of dry steam.

In some embodiments, the composition off-gasses the molecule at a reference vaporization rate in the absence of the bombarding; the vaporized molecule recondenses onto the composition at a reference condensation rate in the absence of the bombarding; converting the molecule into the vaporized molecule in the absence of the bombarding occurs at a reference mass transfer rate, which is equal to the reference vaporization rate minus the reference condensation rate; and the mass transfer rate is at least 100 percent greater than the reference mass transfer rate. In some specific embodiments, the mass transfer rate is at least 10 times greater than the reference mass transfer rate. In some very specific embodiments, the mass transfer rate is at least 100 times greater than the reference mass transfer rate.

In some embodiments, the method has a rate-limiting step; the mass transfer rate is not the rate-limiting step; the composition off-gasses the molecule at a reference vaporization rate in the absence of the bombarding; the vaporized molecule recondenses onto the composition at a reference condensation rate in the absence of the bombarding; converting the molecule into the vaporized molecule in the absence of the bombarding occurs at a reference mass transfer rate, which is equal to the reference vaporization rate minus the reference condensation rate; the method has a reference rate-limiting step in the absence of the bombarding; and the reference mass transfer rate is the reference rate-limiting step.

In some embodiments, bombarding the composition with a sweep gas comprises turbulent flow of the sweep gas.

In some embodiments, the sweep gas has a Reynolds number of at least 1 during the bombarding.

In some embodiments, the sweep gas has a Reynolds number of no greater than 100,000 during the bombarding.

In some embodiments, the composition has a drag coefficient of at least 0.5 when the composition is bombarded with the sweep gas.

In some embodiments, the method comprises processing a starting composition to increase its surface-area-to-volume ratio, wherein providing the composition comprises the processing.

In some embodiments, providing the composition comprises preparing the composition from a starting composition; the starting composition has a surface-area-to-volume ratio that is less than the surface-area-to-volume ratio of the composition; and the processing comprises one or both of increasing the surface-area-to-volume ratio of the starting composition and selecting a portion of the starting composition that has a greater surface-area-to-volume ratio than the rest of the starting composition.

In some embodiments, providing the composition comprises one or both of grinding a starting composition and separating the starting composition by size.

In some embodiments, providing the composition comprises selecting particles of a starting composition that have a particle size of no greater than 5 millimeters.

In some embodiments, providing the composition comprises grinding a starting composition to an average particle size that is no greater than 5 millimeters.

In some embodiments, the surface-area-to-volume ratio of the composition supports a vaporization rate of at least 5 micrograms of the molecule per gram of the composition per second at the temperature and the pressure of the gas phase.

In some embodiments, the surface-area-to-volume ratio of the composition supports a mass transfer rate of at least 5 micrograms of the molecule per gram of the composition per second at the temperature and the pressure of the gas phase.

In some embodiments, the composition has a surface-area-to-volume ratio that is greater than 500 per meter. In some specific embodiments, the composition has a surface-area-to-volume ratio of at least 1000 per meter. In some even more specific embodiments, the composition has a surface-area-to-volume ratio of at least 2400 per meter. In some even more specific embodiments, the composition has a surface-area-to-volume ratio of at least 5000 per meter. In some even more specific embodiments, the composition has a surface-area-to-volume ratio of at least 10,000 per meter. In some very specific embodiments, the composition has a surface-area-to-volume ratio of at least 24,000 per meter.

In some embodiments, a reference composition is identical to the composition except that the reference composition has a surface-area-to-volume ratio of less than 500 per meter; the reference composition off-gasses the molecule at a reference vaporization rate when the method is performed on the reference composition; the vaporized molecule off-gassed by the reference composition recondenses onto the reference composition at a reference condensation rate when the method is performed on the reference composition; the reference composition has a reference mass transfer rate, which is equal to the reference vaporization rate minus the reference condensation rate; and the mass transfer rate is at least 100 percent greater than the reference mass transfer rate. In some specific embodiments, the mass transfer rate is at least 10 times greater than the reference mass transfer rate. In some very specific embodiments, the mass transfer rate is at least 100 times greater than the reference mass transfer rate.

In some embodiments, the method has a rate-limiting step when the method is performed with the composition; the mass transfer rate is not the rate-limiting step; when the method is performed on a reference composition that is identical to the composition except that the reference composition has a surface-area-to-volume ratio of less than 500 per meter, then the reference composition off-gasses the vaporized molecule at a reference vaporization rate; when the method is performed on the reference composition, then the vaporized molecule off-gassed by the reference composition recondenses onto the reference composition at a reference condensation rate; the reference composition has a reference mass transfer rate, which is equal to the reference vaporization rate minus the reference condensation rate; the method has a reference rate-limiting step when the method is performed with the reference composition; and the reference mass transfer rate is the reference rate-limiting step.

In some embodiments, the composition has an average terminal velocity of no greater than 5 meters per second in still, dry air at 1 atmosphere of pressure.

In some embodiments, providing the composition comprises selecting a portion of a starting composition that has a terminal velocity of no greater than 5 meters per second in still, dry air at 1 atmosphere of pressure.

In some embodiments, the composition has an average terminal velocity of no greater than 1 meter per second in still, dry air at 1 atmosphere of pressure.

In some embodiments, providing the composition comprises selecting a portion of a starting composition that has a terminal velocity of no greater than 1 meter per second in still, dry air at 1 atmosphere of pressure.

In some embodiments, the method comprises suspending at least 75 percent of the composition in the gas phase, and the energy transfer is performed while the at least 75 percent of the composition is suspended in the gas phase. In some specific embodiments, the method comprises suspending at least 98 percent of the composition in the gas phase, and the energy transfer is performed while the at least 98 percent of the composition is suspended in the gas phase.

In some embodiments, the residual solids and liquids comprise a monosaccharide, disaccharide, or polysaccharide.

In some embodiments, the residual solids and liquids comprise cellulose. In some specific embodiments, the residual solids and liquids comprise cellulose I.

In some embodiments, the residual solids and liquids comprise hemicellulose.

In some embodiments, the residual solids and liquids comprise residual pentose.

In some embodiments, the residual solids and liquids comprise residual phenolic molecules.

In some embodiments, the residual solids and liquids comprise residual monolignols, lignans, and lignin.

In some embodiments, the residual solids and liquids comprise an amino acid, polypeptide, or protein.

In some embodiments, the residual solids and liquids comprise a nucleobase, nucleoside, nucleotide, or nucleic acid.

In some embodiments, the residual solids and liquids comprise a triglyceride.

In some embodiments, the residual solids and liquids comprise a wax.

In some embodiments, the residual solids and liquids comprise chlorophyll.

In some embodiments, the residual solids and liquids comprise sodium ion, potassium ion, calcium ion, iron(II), iron(III), magnesium ion, or phosphate.

In some embodiments, the composition comprises biological cells; the biological cells have cell walls; and the method comprises generating sufficient pressure within the biological cells to rupture at least 10 percent of the cell walls.

In some embodiments, the composition comprises biological cells; the biological cells have cell walls; the method comprises vaporizing an accessory molecule within the biological cells; and vaporizing the accessory molecule generates sufficient pressure within the biological cells to rupture at least 10 percent of the cell walls.

In some embodiments, the composition comprises biological cells; the biological cells have cell walls; and the method comprises vaporizing an accessory molecule within the biological cells at a rate sufficient to generate pressure within the biological cells that ruptures at least 10 percent of the cell walls.

In some embodiments, the method comprises vaporizing the accessory molecule and rupturing the cell walls in a total time of no greater than 60 seconds.

In some embodiments, the composition comprises the accessory molecule at a concentration of at least 1000 parts per million by mass.

In some embodiments, the composition comprises the accessory molecule at a concentration of no greater than 20 percent by mass.

In some embodiments, the accessory molecule has a boiling point at the pressure of the gas phase that is less than the temperature of the gas phase.

In some embodiments, the accessory molecule is water.

In some embodiments, the method comprises combining the accessory molecule and a starting composition, wherein providing the composition comprises the combining.

In some embodiments, the accessory molecule is the molecule.

In some embodiments, the method comprises generating sufficient pressure within the biological cells to rupture at least 40 percent of the cell walls. In some specific embodiments, the method comprises generating sufficient pressure within the biological cells to rupture at least 75 percent of the cell walls.

In some embodiments, the composition off-gasses the vaporized molecule at a vaporization rate; and the rupturing increases the vaporization rate.

In some embodiments, the rupturing increases the vaporization rate to at least 5 micrograms of the molecule per gram of the composition per second.

In some embodiments, the rupturing increases the mass transfer rate. In some specific embodiments, the rupturing increases the mass transfer rate to at least 5 micrograms of the molecule per gram of the composition per second.

In some embodiments, the composition comprises the molecule at a concentration of at least 10 parts per million by mass.

In some embodiments, the composition comprises the molecule at a concentration of no greater than 20 percent by mass.

In some embodiments, the composition comprises the molecule at a concentration of at least 10 parts per million and no greater than 1 percent by mass.

In some embodiments, the boiling point of the molecule at the pressure of the gas phase is at least 10 percent greater than the boiling point of water in Celsius at the pressure of the gas phase. In some specific embodiments, the boiling point of the molecule at the pressure of the gas phase is at least 25 percent greater than the boiling point of water in Celsius at the pressure of the gas phase. In some very specific embodiments, the boiling point of the molecule at the pressure of the gas phase is at least 50 percent greater than the boiling point of water in Celsius at the pressure of the gas phase.

In some embodiments, the boiling point of the molecule at the pressure of the gas phase is greater than 100 degrees Celsius.

In some embodiments, the temperature of the gas phase is greater than the boiling point of water at the pressure of the gas phase. In some specific embodiments, the temperature of the gas phase is at least 100 percent greater than the boiling point of water in Celsius at the pressure of the gas phase.

In some embodiments, the temperature of the gas phase is less than 250 degrees Celsius.

In some embodiments, the pressure of the gas phase is no greater than the vapor pressure of water at the temperature of the gas phase.

In some embodiments, the pressure of the gas phase is at least 0.1 atmospheres.

In some embodiments, the pressure of the gas phase is no greater than 100 atmospheres.

In some embodiments, the pressure of the gas phase is at least 0.5 atmospheres and no greater than 2 atmospheres. In some specific embodiments, the pressure of the gas phase is at least 0.75 atmospheres and no greater than 1.25 atmospheres.

In some embodiments, the method comprises converting at least 10 percent of the molecule into a condensed molecule of the distillate by mole. In some specific embodiments, the method comprises converting at least 25 percent of the molecule into a condensed molecule of the distillate by mole. In some very specific embodiments, the method comprises converting at least 60 percent of the molecule into a condensed molecule of the distillate by mole.

In some embodiments, the composition comprises a starting concentration of the molecule by mass; the distillate comprises an ending concentration of the molecule by mass; and the ending concentration is at least 5 times greater than the starting concentration. In some specific embodiments, the composition comprises a starting concentration of the molecule by mass; the distillate comprises an ending concentration of the molecule by mass; and the ending concentration is at least 50 times greater than the starting concentration. In some very specific embodiments, the composition comprises a starting concentration of the molecule by mass; the distillate comprises an ending concentration of the molecule by mass; and the ending concentration is at least 500 times greater than the starting concentration.

In some embodiments, condensing the portion of the vapor comprises increasing the pressure of the gas phase, reducing the temperature of the gas phase, or both increasing the pressure of the gas phase and reducing the temperature of the gas phase.

In some embodiments, condensing the portion of the vapor comprises contacting the vapor with a heat sink.

In some embodiments, the method comprises converting the molecule into the vaporized molecule in a system that contains the gas phase, wherein the system is configured to inhibit the gas phase from escaping the system.

In some embodiments, the method comprises providing a system comprising a first chamber and a second chamber, wherein converting the composition into the vapor and the residual solids and liquids is performed in the first chamber; the first chamber and the second chamber are in fluid communication such that the portion of the vapor can be directed into the second chamber; the method comprises directing the portion of the vapor from the first chamber to the second chamber; and condensing the portion of the vapor is performed in a second chamber.

In some embodiments, the system allows passage of the vaporized molecule from the first chamber to the second chamber.

In some embodiments, the system allows passage of the gas phase from the first chamber to the second chamber.

In some embodiments, the system inhibits passage of the residual solids and liquids from the first chamber to the second chamber.

In some embodiments, the system inhibits passage of the composition from the first chamber to the second chamber.

In some embodiments, the system inhibits passage of solids from the first chamber to the second chamber.

In some embodiments, the system inhibits passage of liquids from the first chamber to the second chamber.

In some embodiments, the system allows passage of gases from the second chamber to the first chamber.

In some embodiments, the method comprises condensing the vaporized molecule into a condensed molecule of the distillate from a first portion of the composition in the second chamber and concurrently converting the molecule into the vaporized molecule from a subsequent portion of the composition in the first chamber.

In some embodiments, the method comprises feeding the composition into the first chamber of the system at a feed rate, which is the amount of the molecule that is fed into the first chamber per unit time; converting the molecule into the vaporized molecule at a mass transfer rate, which is the amount of the molecule that the composition off-gases minus the amount of the vaporized molecule that recondenses onto the composition per unit time; and condensing the vaporized molecule into a condensed molecule of the distillate at a collection rate, which is the amount of the vaporized molecule that is condensed into the condensed molecule per unit time, wherein the method is performed such that the collection rate is at least 50 percent and no greater than 100 percent of the mass transfer rate per mole over a period of time; the mass transfer rate is at least 50 percent and no greater than 100 percent of the feed rate per mole over a concurrent period of time; and the period of time is chronologically identical to the concurrent period of time. In some embodiments, the period of time and the concurrent period of time are the same 10 second period. In some specific embodiments, the period of time and the concurrent period of time are the same 5 second period. In some very specific embodiments, the period of time and the concurrent period of time are the same 1 second period.

In some embodiments, the composition comprises hemicellulose.

In some embodiments, the composition comprises biomass of a plant.

In some embodiments, the composition comprises biomass of a perennial plant.

In some embodiments, the composition comprises wood.

In some embodiments, the composition comprises sawdust.

In some embodiments, the composition comprises heartwood.

In some specific embodiments, the composition comprises coniferous wood. In some very specific embodiments, the composition comprises biomass from Araucaria; hoop pine (Araucaria cunninghamii); monkey puzzle tree (Araucaria araucana); Paraná pine (Araucaria angustifolia); cedar (Cedrus); celery-top pine (Phyllocladus aspleniifolius); cypress; Arizona cypress (Cupressus arizonica); bald cypress (Taxodium distichum); alerce (Fitzroya cupressoides); Hinoki cypress (Chamaecyparis obtusa); Lawson's cypress (Chamaecyparis lawsoniana); Mediterranean cypress (Cupressus sempervirens); Douglas fir (Pseudotsuga menziesii); European yew (Taxus baccata); fir (Abies); balsam fir (Abies balsamea); silver fir (Abies alba); noble fir (Abies procera); Pacific silver fir (Abies amabilis); hemlock (Tsuga); eastern hemlock (Tsuga canadensis); mountain hemlock (Tsuga mertensiana); western hemlock (Tsuga heterophylla); Huon pine (Lagarostrobos franklinii); kauri (Agathis australis); Queensland kauri (Agathis robusta); Japanese nutmeg-yew (Torreya nucifera); larch (Larix); European larch (Larix decidua); Japanese larch (Larix kaempferi); tamarack (Larix laricina); western larch (Larix occidentalis); pine (Pinus); European black pine (Pinus nigra); jack pine (Pinus banksiana); lodgepole pine (Pinus contorta); Monterey pine (Pinus radiata); Ponderosa pine (Pinus ponderosa); red pine (Pinus resinosa); Scots pine (Pinus sylvestris); white pine; eastern white pine (Pinus strobus); western white pine (Pinus monticola); sugar pine (Pinus lambertiana); southern yellow pine; loblolly pine (Pinus taeda); longleaf pine (Pinus palustris); pitch pine (Pinus rigida); shortleaf pine (Pinus echinata); red cedar; eastern red cedar (Juniperus virginiana); western red cedar (Thuja plicata); coast redwood (Sequoia sempervirens); rimu (Dacrydium cupressinum); spruce (Picea); Norway spruce (Picea abies); black spruce (Picea mariana); red spruce (Picea rubens); Sitka spruce (Picea sitchensis); white spruce (Picea glauca); sugi (Cryptomeria japonica); white cedar; northern white cedar (Thuja occidentalis); Atlantic white cedar (Chamaecyparis thyoides); or nootka cypress (Cupressus nootkatensis).

In some embodiments, the composition comprises biomass from Araucaria.

In some embodiments, the composition comprises biomass from hoop pine (Araucaria cunninghamii).

In some embodiments, the composition comprises biomass from monkey puzzle tree (Araucaria araucana).

In some embodiments, the composition comprises biomass from Paraná pine (Araucaria angustifolia).

In some embodiments, the composition comprises biomass from cedar (Cedrus).

In some embodiments, the composition comprises biomass from celery-top pine (Phyllocladus aspleniifolius).

In some embodiments, the composition comprises biomass from cypress.

In some embodiments, the composition comprises biomass from Arizona cypress (Cupressus arizonica).

In some embodiments, the composition comprises biomass from bald cypress (Taxodium distichum).

In some embodiments, the composition comprises biomass from alerce (Fitzroya cupressoides).

In some embodiments, the composition comprises biomass from Hinoki cypress (Chamaecyparis obtusa).

In some embodiments, the composition comprises biomass from Lawson's cypress (Chamaecyparis lawsoniana).

In some embodiments, the composition comprises biomass from Mediterranean cypress (Cupressus sempervirens).

In some embodiments, the composition comprises biomass from Douglas fir (Pseudotsuga menziesii).

In some embodiments, the composition comprises biomass from European yew (Taxus baccata).

In some embodiments, the composition comprises biomass from fir (Abies).

In some embodiments, the composition comprises biomass from balsam fir (Abies balsamea).

In some embodiments, the composition comprises biomass from silver fir (Abies alba).

In some embodiments, the composition comprises biomass from noble fir (Abies procera).

In some embodiments, the composition comprises biomass from Pacific silver fir (Abies amabilis).

In some embodiments, the composition comprises biomass from hemlock (Tsuga).

In some embodiments, the composition comprises biomass from eastern hemlock (Tsuga canadensis).

In some embodiments, the composition comprises biomass from mountain hemlock (Tsuga mertensiana).

In some embodiments, the composition comprises biomass from western hemlock (Tsuga heterophylla).

In some embodiments, the composition comprises biomass from Huon pine (Lagarostrobos franklinii).

In some embodiments, the composition comprises biomass from kauri (Agathis australis).

In some embodiments, the composition comprises biomass from Queensland kauri (Agathis robusta).

In some embodiments, the composition comprises biomass from Japanese nutmeg-yew (Torreya nucifera).

In some embodiments, the composition comprises biomass from larch (Larix).

In some embodiments, the composition comprises biomass from European larch (Larix decidua).

In some embodiments, the composition comprises biomass from Japanese larch (Larix kaempferi).

In some embodiments, the composition comprises biomass from tamarack (Larix laricina).

In some embodiments, the composition comprises biomass from western larch (Larix occidentalis).

In some embodiments, the composition comprises biomass from pine (Pinus).

In some embodiments, the composition comprises biomass from European black pine (Pinus nigra).

In some embodiments, the composition comprises biomass from jack pine (Pinus banksiana).

In some embodiments, the composition comprises biomass from lodgepole pine (Pinus contorta).

In some embodiments, the composition comprises biomass from Monterey pine (Pinus radiata).

In some embodiments, the composition comprises biomass from Ponderosa pine (Pinus ponderosa).

In some embodiments, the composition comprises biomass from red pine (Pinus resinosa).

In some embodiments, the composition comprises biomass from Scots pine (Pinus sylvestris).

In some embodiments, the composition comprises biomass from white pine.

In some embodiments, the composition comprises biomass from eastern white pine (Pinus strobus).

In some embodiments, the composition comprises biomass from western white pine (Pinus monticola).

In some embodiments, the composition comprises biomass from sugar pine (Pinus lambertiana).

In some embodiments, the composition comprises biomass from southern yellow pine.

In some embodiments, the composition comprises biomass from loblolly pine (Pinus taeda).

In some embodiments, the composition comprises biomass from longleaf pine (Pinus palustris).

In some embodiments, the composition comprises biomass from pitch pine (Pinus rigida).

In some embodiments, the composition comprises biomass from shortleaf pine (Pinus echinata).

In some embodiments, the composition comprises biomass from red cedar.

In some embodiments, the composition comprises biomass from eastern red cedar (Juniperus virginiana).

In some embodiments, the composition comprises biomass from western red cedar (Thuja plicata).

In some embodiments, the composition comprises biomass from coast redwood (Sequoia sempervirens).

In some embodiments, the composition comprises biomass from rimu (Dacrydium cupressinum).

In some embodiments, the composition comprises biomass from spruce (Picea).

In some embodiments, the composition comprises biomass from Norway spruce (Picea abies).

In some embodiments, the composition comprises biomass from black spruce (Picea mariana).

In some embodiments, the composition comprises biomass from red spruce (Picea rubens).

In some embodiments, the composition comprises biomass from Sitka spruce (Picea sitchensis).

In some embodiments, the composition comprises biomass from white spruce (Picea glauca).

In some embodiments, the composition comprises biomass from sugi (Cryptomeria japonica).

In some embodiments, the composition comprises biomass from white cedar.

In some embodiments, the composition comprises biomass from northern white cedar (Thuja occidentalis).

In some embodiments, the composition comprises biomass from Atlantic white cedar (Chamaecyparis thyoides).

In some embodiments, the composition comprises biomass from nootka cypress (Cupressus nootkatensis).

In some specific embodiments, the composition comprises angiosperm wood. In some very specific embodiments, the composition comprises biomass from abachi (Triplochiton scleroxylon); acacia; African padauk (Pterocarpus soyauxii); afzelia (Afzelia africana); agba (Gossweilerodendron balsamiferum); alder (Alnus); black alder (Alnus glutinosa); red alder (Alnus rubra); ash (Fraxinus); black ash (Fraxinus nigra); blue ash (Fraxinus quadrangulata); common ash (Fraxinus excelsior); green ash (Fraxinus pennsylvanica); Oregon ash (Fraxinus latifolia); pumpkin ash (Fraxinus profunda); white ash (Fraxinus americana); aspen (Populus); bigtooth aspen (Populus gradidentata); European aspen (Populus tremula); quaking aspen (Populus tremuloides); Australian red cedar (Toona ciliata); ayan (Distemonanthus benthamianus); balsa (Ochroma pyramidale); basswood; American basswood (Tilia americana); white basswood (Tilia heterophylla); American beech (Fagus grandifolia); birch (Betula); gray birch (Betula populifolia); black birch (Betula nigra); paper birch (Betula papyrifera); sweet birch (Betula lenta); yellow birch (Betula alleghaniensis); silver birch (Betula pendula); downy birch (Betula pubescens); blackbean (Castanospermum australe); blackwood; Australian blackwood (Acacia melanoxylon); African blackwood (Dalbergia melanoxylon); bloodwood (Brosimum rubescens); boxelder (Acer negundo); boxwood (Buxus sempervirens); Brazilian walnut (Ocotea porosa); brazilwood (Caesalpinia echinata); buckeye (Aesculus); horse-chestnut (Aesculus hippocastanum); Ohio buckeye (Aesculus glabra); yellow buckeye (Aesculus flava); butternut (Juglans cinerea); California bay laurel (Umbellularia californica); camphor tree (Cinnamomum camphora); cape chestnut (Calodendrum capense); catalpa (Catalpa); Ceylon satinwood (Chloroxylon swietenia); cherry (Prunus); black cherry (Prunus serotina); red cherry (Prunus pensylvanica); wild cherry (Prunus avium); chestnut (Castanea); chestnut (Castanea sativa); American chestnut (Castanea dentata); coachwood (Ceratopetalum apetalum); cocobolo (Dalbergia retusa); corkwood (Leitneria floridana); cottonwood; eastern cottonwood (Populus deltoides); swamp cottonwood (Populus heterophylla); cucumbertree (Magnolia acuminata); cumaru (Dipteryx); dogwood (Cornus); flowering dogwood (Cornus florida); Pacific dogwood (Cornus nuttallii); ebony (Diospyros); Andaman marblewood (Diospyros kurzii); ebène marbre (Diospyros melanida); African ebony (Diospyros crassiflora); Ceylon ebony (Diospyros ebenum); elm; American elm (Ulmus americana); English elm (Ulmus procera); rock elm (Ulmus thomasii); red elm (Ulmus rubra); wych elm (Ulmus glabra); eucalyptus; flooded gum (Eucalyptus grandis); white mahogany (Eucalyptus acmenoides); brown mallet (Eucalyptus astringens); southern mahogany (Eucalyptus botryoides); river red gum (Eucalyptus camaldulensis); karri (Eucalyptus diversicolor); blue gum (Eucalyptus globulus); rose gum (Eucalyptus grandis); york gum (Eucalyptus loxophleba); jarrah (Eucalyptus marginata); tallowwood (Eucalyptus microcorys); grey ironbark (Eucalyptus paniculata); blackbutt (Eucalyptus pilularis); mountain ash (Eucalyptus regnans); Australian oak (Eucalyptus obliqua); alpine ash (Eucalyptus delegatensis); red mahogany (Eucalyptus resinifera); swamp mahogany (Eucalyptus robusta); Sydney blue gum (Eucalyptus saligna); red ironbark (Eucalyptus sideroxylon); redwood (Eucalyptus transcontinentalis); Wandoo (Eucalyptus wandoo); European crabapple (Malus sylvestris); European pear (Pyrus communis); tigerwood (Astronium); greenheart (Chlorocardium rodiei); mpingo (Dalbergia melanoxylon); guanandi (Calophyllum brasiliense); gum (Eucalyptus); gumbo limbo (Bursera simaruba); hackberry (Celtis occidentalis); hickory (Carya); pecan (Carya illinoinensis); pignut hickory (Carya glabra); shagbark hickory (Carya ovata); shellbark hickory (Carya laciniosa); hornbeam (Carpinus); American hophornbeam (Ostrya virginiana); ipê (Handroanthus); African teak (Milicia excelsa); ironwood; balau (Shorea); American hornbeam (Carpinus caroliniana); sheoak (Casuarina equisetifolia); giant ironwood (Choricarpia subargentea); diesel tree (Copaifera langsdorffii); Borneo ironwood (Eusideroxylon zwageri); lignum vitae; guaiacwood (Guaiacum officinale); holywood (Guaiacum sanctum); takian (Hopea odorata); black ironwood (Krugiodendron ferreum); black ironwood (Olea); Lebombo ironwood (Androstachys johnsonii); Catalina ironwood (Lyonothamnus floribundus); Ceylon ironwood (Mesua ferrea); desert ironwood (Olneya tesota); Persian ironwood (Parrotia persica); Brazilian ironwood (Caesalpinia ferrea); yellow lapacho (Tabebuia serratifolia); jacarandá-boca-de-sapo (Jacaranda brasiliana); jacarandá de Brasil (Dalbergia nigra); jatobá (Hymenaea courbaril); kingwood (Dalbergia cearensis); lacewood; northern silky oak (Cardwellia sublimis); American sycamore (Platanus occidentalis); London plane (Platanus x acerifolia); limba (Terminalia superba); locust; black locust (Robinia pseudoacacia); honey locust (Gleditsia triacanthos); mahogany; genuine mahogany (Swietenia); West Indies mahogany (Swietenia mahagoni); bigleaf mahogany (Swietenia macrophylla); Pacific Coast mahogany (Swietenia humilis); African mahogany (Khaya); Chinese mahogany (Toona sinensis); Australian red cedar (Toona ciliata); Philippine mahogany (Toona calantas); Indonesian mahogany (Toona sureni); sapele (Entandrophragma cylindricum); sipo (Entandrophragma utile); tiama (Entandrophragma angolense); kosipo (Entandrophragma candollei); mountain mahogany (Entandrophragma caudatumi); Indian mahogany (Chukrasia velutina); Spanish Cedar (Cedrela odorata); light bosse (Guarea cedrata); dark bosse (Guarea thompsonii); American muskwood (Guarea grandifolia); carapa (Carapa guianensis); bead-tree (Melia azedarach); maple (Acer); hard maple; sugar maple (Acer saccharum); black maple (Acer nigrum); soft maple; boxelder (Acer negundo); red maple (Acer rubrum); silver maple (Acer saccharinum); European maple; sycamore maple (Acer pseudoplatanus); marblewood (Marmaroxylon racemosum); marri (Corymbia calophylla); meranti (Shorea); merbau (Intsia bijuga); mopane (Colophospermum mopane); oak (Quercus); white oak (Quercus alba); bur oak (Quercus macrocarpa); post oak (Quercus stellata); swamp white oak (Quercus bicolor); southern live oak (Quercus virginiana); swamp chestnut oak (Quercus michauxii); chestnut oak (Quercus prinus); chinkapin oak (Quercus muhlenbergii); canyon live oak (Quercus chrysolepis); overcup oak (Quercus lyrata); English oak (Quercus robur); red oak; northern red oak (Quercus rubra); eastern black oak (Quercus velutina); laurel oak (Quercus laurifolia); southern red oak (Quercus falcata); water oak (Quercus nigra); willow oak (Quercus phellos); Nuttall's oak (Quercus texana); okoumé (Aucoumea klaineana); olive (Olea europaea); pink ivory (Berchemia zeyheri); poplar; balsam poplar (Populus balsamifera); black poplar (Populus nigra); hybrid black poplar (Populus x canadensis); purpleheart (Peltogyne); Queensland maple (Flindersia brayleyana); Queensland walnut (Endiandra palmerstonii); ramin (Gonystylus); redheart, chakté-coc (Erythroxylon mexicanum); sal (Shorea robusta); sweetgum (Liquidambar styraciflua); sandalwood (Santalum); Australian sandalwood (Santalum spicatum); Indian sandalwood (Santalum album); Hawaiian sandalwood (Santalum ellipticum, Santalum freycinetianum, Santalum paniculatum, Santalum haleakalae); Santalum acuminatum; Santalum yasi; Santalum spicatum; sassafras (Sassafras albidum); southern sassafras (Atherosperma moschatum); satiné (Brosimum rubescens); silky oak (Grevillea robusta); silver wattle (Acacia dealbata); sourwood (Oxydendrum arboreum); Spanish-cedar (Cedrela odorata); Spanish elm (Cordia alliodora); tamboti (Spirostachys africana); teak (Tectona grandis); Thailand rosewood (Dalbergia cochinchinensis); tupelo (Nyssa); black tupelo (Nyssa sylvatica); tulip tree (Liriodendron tulipifera); turpentine (Syncarpia glomulifera); walnut (Juglans); Eastern black walnut (Juglans nigra); common walnut (Juglans regia); wenge (Millettia laurentii); panga-panga (Millettia stuhlmannii); willow (Salix); black willow (Salix nigra); cricket-bat willow (Salix alba Caerulea); white willow (Salix alba); weeping willow (Salix babylonica); or zingana (Microberlinia brazzavillensis).

In some embodiments, the composition comprises biomass from abachi (Triplochiton scleroxylon).

In some embodiments, the composition comprises biomass from acacia.

In some embodiments, the composition comprises biomass from African padauk (Pterocarpus soyauxii).

In some embodiments, the composition comprises biomass from afzelia (Afzelia africana).

In some embodiments, the composition comprises biomass from agba (Gossweilerodendron balsamiferum).

In some embodiments, the composition comprises biomass from alder (Alnus).

In some embodiments, the composition comprises biomass from black alder (Alnus glutinosa).

In some embodiments, the composition comprises biomass from red alder (Alnus rubra).

In some embodiments, the composition comprises biomass from ash (Fraxinus).

In some embodiments, the composition comprises biomass from black ash (Fraxinus nigra).

In some embodiments, the composition comprises biomass from blue ash (Fraxinus quadrangulata).

In some embodiments, the composition comprises biomass from common ash (Fraxinus excelsior).

In some embodiments, the composition comprises biomass from green ash (Fraxinus pennsylvanica).

In some embodiments, the composition comprises biomass from Oregon ash (Fraxinus latifolia).

In some embodiments, the composition comprises biomass from pumpkin ash (Fraxinus profunda).

In some embodiments, the composition comprises biomass from white ash (Fraxinus americana).

In some embodiments, the composition comprises biomass from aspen (Populus).

In some embodiments, the composition comprises biomass from bigtooth aspen (Populus gradidentata).

In some embodiments, the composition comprises biomass from European aspen (Populus tremula).

In some embodiments, the composition comprises biomass from quaking aspen (Populus tremuloides).

In some embodiments, the composition comprises biomass from Australian red cedar (Toona ciliata).

In some embodiments, the composition comprises biomass from ayan (Distemonanthus benthamianus).

In some embodiments, the composition comprises biomass from balsa (Ochroma pyramidale).

In some embodiments, the composition comprises biomass from basswood.

In some embodiments, the composition comprises biomass from American basswood (Tilia americana).

In some embodiments, the composition comprises biomass from white basswood (Tilia heterophylla).

In some embodiments, the composition comprises biomass from American beech (Fagus grandifolia).

In some embodiments, the composition comprises biomass from birch (Betula).

In some embodiments, the composition comprises biomass from gray birch (Betula populifolia).

In some embodiments, the composition comprises biomass from black birch (Betula nigra).

In some embodiments, the composition comprises biomass from paper birch (Betula papyrifera).

In some embodiments, the composition comprises biomass from sweet birch (Betula lenta).

In some embodiments, the composition comprises biomass from yellow birch (Betula alleghaniensis).

In some embodiments, the composition comprises biomass from silver birch (Betula pendula).

In some embodiments, the composition comprises biomass from downy birch (Betula pubescens).

In some embodiments, the composition comprises biomass from blackbean (Castanospermum australe).

In some embodiments, the composition comprises biomass from blackwood.

In some embodiments, the composition comprises biomass from Australian blackwood (Acacia melanoxylon).

In some embodiments, the composition comprises biomass from African blackwood (Dalbergia melanoxylon).

In some embodiments, the composition comprises biomass from bloodwood (Brosimum rubescens).

In some embodiments, the composition comprises biomass from boxelder (Acer negundo).

In some embodiments, the composition comprises biomass from boxwood (Buxus sempervirens).

In some embodiments, the composition comprises biomass from Brazilian walnut (Ocotea porosa).

In some embodiments, the composition comprises biomass from brazilwood (Caesalpinia echinata).

In some embodiments, the composition comprises biomass from buckeye (Aesculus).

In some embodiments, the composition comprises biomass from horse-chestnut (Aesculus hippocastanum).

In some embodiments, the composition comprises biomass from Ohio buckeye (Aesculus glabra).

In some embodiments, the composition comprises biomass from yellow buckeye (Aesculus flava).

In some embodiments, the composition comprises biomass from butternut (Juglans cinerea).

In some embodiments, the composition comprises biomass from California bay laurel (Umbellularia californica).

In some embodiments, the composition comprises biomass from camphor tree (Cinnamomum camphora).

In some embodiments, the composition comprises biomass from cape chestnut (Calodendrum capense).

In some embodiments, the composition comprises biomass from catalpa (Catalpa).

In some embodiments, the composition comprises biomass from Ceylon satinwood (Chloroxylon swietenia).

In some embodiments, the composition comprises biomass from cherry (Prunus).

In some embodiments, the composition comprises biomass from black cherry (Prunus serotina).

In some embodiments, the composition comprises biomass from red cherry (Prunus pensylvanica).

In some embodiments, the composition comprises biomass from wild cherry (Prunus avium).

In some embodiments, the composition comprises biomass from chestnut (Castanea).

In some embodiments, the composition comprises biomass from chestnut (Castanea sativa).

In some embodiments, the composition comprises biomass from American chestnut (Castanea dentata).

In some embodiments, the composition comprises biomass from coachwood (Ceratopetalum apetalum).

In some embodiments, the composition comprises biomass from cocobolo (Dalbergia retusa).

In some embodiments, the composition comprises biomass from corkwood (Leitneria floridana).

In some embodiments, the composition comprises biomass from cottonwood.

In some embodiments, the composition comprises biomass from eastern cottonwood (Populus deltoides).

In some embodiments, the composition comprises biomass from swamp cottonwood (Populus heterophylla).

In some embodiments, the composition comprises biomass from cucumbertree (Magnolia acuminata).

In some embodiments, the composition comprises biomass from cumaru (Dipteryx).

In some embodiments, the composition comprises biomass from dogwood (Cornus).

In some embodiments, the composition comprises biomass from flowering dogwood (Cornus florida).

In some embodiments, the composition comprises biomass from Pacific dogwood (Cornus nuttallii).

In some embodiments, the composition comprises biomass from ebony (Diospyros).

In some embodiments, the composition comprises biomass from Andaman marblewood (Diospyros kurzii).

In some embodiments, the composition comprises biomass from ebène marbre (Diospyros melanida).

In some embodiments, the composition comprises biomass from African ebony (Diospyros crassiflora).

In some embodiments, the composition comprises biomass from Ceylon ebony (Diospyros ebenum).

In some embodiments, the composition comprises biomass from elm.

In some embodiments, the composition comprises biomass from American elm (Ulmus americana).

In some embodiments, the composition comprises biomass from English elm (Ulmus procera).

In some embodiments, the composition comprises biomass from rock elm (Ulmus thomasii).

In some embodiments, the composition comprises biomass from red elm (Ulmus rubra).

In some embodiments, the composition comprises biomass from wych elm (Ulmus glabra).

In some embodiments, the composition comprises biomass from eucalyptus.

In some embodiments, the composition comprises biomass from flooded gum (Eucalyptus grandis).

In some embodiments, the composition comprises biomass from white mahogany (Eucalyptus acmenoides).

In some embodiments, the composition comprises biomass from brown mallet (Eucalyptus astringens).

In some embodiments, the composition comprises biomass from southern mahogany (Eucalyptus botryoides).

In some embodiments, the composition comprises biomass from river red gum (Eucalyptus camaldulensis).

In some embodiments, the composition comprises biomass from karri (Eucalyptus diversicolor).

In some embodiments, the composition comprises biomass from blue gum (Eucalyptus globulus).

In some embodiments, the composition comprises biomass from rose gum (Eucalyptus grandis).

In some embodiments, the composition comprises biomass from york gum (Eucalyptus loxophleba).

In some embodiments, the composition comprises biomass from jarrah (Eucalyptus marginata).

In some embodiments, the composition comprises biomass from tallowwood (Eucalyptus microcorys).

In some embodiments, the composition comprises biomass from grey ironbark (Eucalyptus paniculata).

In some embodiments, the composition comprises biomass from blackbutt (Eucalyptus pilularis).

In some embodiments, the composition comprises biomass from mountain ash (Eucalyptus regnans).

In some embodiments, the composition comprises biomass from Australian oak (Eucalyptus obliqua).

In some embodiments, the composition comprises biomass from alpine ash (Eucalyptus delegatensis).

In some embodiments, the composition comprises biomass from red mahogany (Eucalyptus resinifera).

In some embodiments, the composition comprises biomass from swamp mahogany (Eucalyptus robusta).

In some embodiments, the composition comprises biomass from Sydney blue gum (Eucalyptus saligna).

In some embodiments, the composition comprises biomass from red ironbark (Eucalyptus sideroxylon).

In some embodiments, the composition comprises biomass from redwood (Eucalyptus transcontinentalis).

In some embodiments, the composition comprises biomass from Wandoo (Eucalyptus wandoo).

In some embodiments, the composition comprises biomass from European crabapple (Malus sylvestris).

In some embodiments, the composition comprises biomass from European pear (Pyrus communis).

In some embodiments, the composition comprises biomass from tigerwood (Astronium).

In some embodiments, the composition comprises biomass from greenheart (Chlorocardium rodiei).

In some embodiments, the composition comprises biomass from mpingo (Dalbergia melanoxylon).

In some embodiments, the composition comprises biomass from guanandi (Calophyllum brasiliense).

In some embodiments, the composition comprises biomass from gum (Eucalyptus).

In some embodiments, the composition comprises biomass from gumbo limbo (Bursera simaruba).

In some embodiments, the composition comprises biomass from hackberry (Celtis occidentalis).

In some embodiments, the composition comprises biomass from hickory (Carya).

In some embodiments, the composition comprises biomass from pecan (Carya illinoinensis).

In some embodiments, the composition comprises biomass from pignut hickory (Carya glabra).

In some embodiments, the composition comprises biomass from shagbark hickory (Carya ovata).

In some embodiments, the composition comprises biomass from shellbark hickory (Carya laciniosa).

In some embodiments, the composition comprises biomass from hornbeam (Carpinus).

In some embodiments, the composition comprises biomass from American hophornbeam (Ostrya virginiana).

In some embodiments, the composition comprises biomass from ipê (Handroanthus).

In some embodiments, the composition comprises biomass from African teak (Milicia excelsa).

In some embodiments, the composition comprises biomass from ironwood.

In some embodiments, the composition comprises biomass from balau (Shorea).

In some embodiments, the composition comprises biomass from American hornbeam (Carpinus caroliniana).

In some embodiments, the composition comprises biomass from sheoak (Casuarina equisetifolia).

In some embodiments, the composition comprises biomass from giant ironwood (Choricarpia subargentea).

In some embodiments, the composition comprises biomass from diesel tree (Copaifera langsdorffii).

In some embodiments, the composition comprises biomass from Borneo ironwood (Eusideroxylon zwageri).

In some embodiments, the composition comprises biomass from lignum vitae.

In some embodiments, the composition comprises biomass from guaiacwood (Guaiacum officinale).

In some embodiments, the composition comprises biomass from holywood (Guaiacum sanctum).

In some embodiments, the composition comprises biomass from takian (Hopea odorata).

In some embodiments, the composition comprises biomass from black ironwood (Krugiodendron ferreum).

In some embodiments, the composition comprises biomass from black ironwood (Olea).

In some embodiments, the composition comprises biomass from Lebombo ironwood (Androstachys johnsonii).

In some embodiments, the composition comprises biomass from Catalina ironwood (Lyonothamnus floribundus).

In some embodiments, the composition comprises biomass from Ceylon ironwood (Mesua ferrea).

In some embodiments, the composition comprises biomass from desert ironwood (Olneya tesota).

In some embodiments, the composition comprises biomass from Persian ironwood (Parrotia persica).

In some embodiments, the composition comprises biomass from Brazilian ironwood (Caesalpinia ferrea).

In some embodiments, the composition comprises biomass from yellow lapacho (Tabebuia serratifolia).

In some embodiments, the composition comprises biomass from jacarandá-boca-de-sapo (Jacaranda brasiliana).

In some embodiments, the composition comprises biomass from jacarandá de Brasil (Dalbergia nigra).

In some embodiments, the composition comprises biomass from jatobá (Hymenaea courbaril).

In some embodiments, the composition comprises biomass from kingwood (Dalbergia cearensis).

In some embodiments, the composition comprises biomass from lacewood.

In some embodiments, the composition comprises biomass from northern silky oak (Cardwellia sublimis).

In some embodiments, the composition comprises biomass from American sycamore (Platanus occidentalis).

In some embodiments, the composition comprises biomass from London plane (Platanus x acerifolia).

In some embodiments, the composition comprises biomass from limba (Terminalia superba).

In some embodiments, the composition comprises biomass from locust.

In some embodiments, the composition comprises biomass from black locust (Robinia pseudoacacia).

In some embodiments, the composition comprises biomass from honey locust (Gleditsia triacanthos).

In some embodiments, the composition comprises biomass from mahogany.

In some embodiments, the composition comprises biomass from genuine mahogany (Swietenia).

In some embodiments, the composition comprises biomass from West Indies mahogany (Swietenia mahagoni).

In some embodiments, the composition comprises biomass from bigleaf mahogany (Swietenia macrophylla).

In some embodiments, the composition comprises biomass from Pacific Coast mahogany (Swietenia humilis).

In some embodiments, the composition comprises biomass from African mahogany (Khaya).

In some embodiments, the composition comprises biomass from Chinese mahogany (Toona sinensis).

In some embodiments, the composition comprises biomass from Australian red cedar (Toona ciliata).

In some embodiments, the composition comprises biomass from Philippine mahogany (Toona calantas).

In some embodiments, the composition comprises biomass from Indonesian mahogany (Toona sureni).

In some embodiments, the composition comprises biomass from sapele (Entandrophragma cylindricum).

In some embodiments, the composition comprises biomass from sipo (Entandrophragma utile).

In some embodiments, the composition comprises biomass from tiama (Entandrophragma angolense).

In some embodiments, the composition comprises biomass from kosipo (Entandrophragma candollei).

In some embodiments, the composition comprises biomass from mountain mahogany (Entandrophragma caudatumi).

In some embodiments, the composition comprises biomass from Indian mahogany (Chukrasia velutina).

In some embodiments, the composition comprises biomass from Spanish Cedar (Cedrela odorata).

In some embodiments, the composition comprises biomass from light bosse (Guarea cedrata).

In some embodiments, the composition comprises biomass from dark bosse (Guarea thompsonii).

In some embodiments, the composition comprises biomass from American muskwood (Guarea grandifolia).

In some embodiments, the composition comprises biomass from carapa (Carapa guianensis).

In some embodiments, the composition comprises biomass from bead-tree (Melia azedarach).

In some embodiments, the composition comprises biomass from maple (Acer).

In some embodiments, the composition comprises biomass from hard maple.

In some embodiments, the composition comprises biomass from sugar maple (Acer saccharum).

In some embodiments, the composition comprises biomass from black maple (Acer nigrum).

In some embodiments, the composition comprises biomass from soft maple.

In some embodiments, the composition comprises biomass from boxelder (Acer negundo).

In some embodiments, the composition comprises biomass from red maple (Acer rubrum).

In some embodiments, the composition comprises biomass from silver maple (Acer saccharinum).

In some embodiments, the composition comprises biomass from European maple.

In some embodiments, the composition comprises biomass from sycamore maple (Acer pseudoplatanus).

In some embodiments, the composition comprises biomass from marblewood (Marmaroxylon racemosum).

In some embodiments, the composition comprises biomass from marri (Corymbia calophylla).

In some embodiments, the composition comprises biomass from meranti (Shorea).

In some embodiments, the composition comprises biomass from merbau (Intsia bijuga).

In some embodiments, the composition comprises biomass from mopane (Colophospermum mopane).

In some embodiments, the composition comprises biomass from oak (Quercus).

In some embodiments, the composition comprises biomass from white oak (Quercus alba).

In some embodiments, the composition comprises biomass from bur oak (Quercus macrocarpa).

In some embodiments, the composition comprises biomass from post oak (Quercus stellata).

In some embodiments, the composition comprises biomass from swamp white oak (Quercus bicolor).

In some embodiments, the composition comprises biomass from southern live oak (Quercus virginiana).

In some embodiments, the composition comprises biomass from swamp chestnut oak (Quercus michauxii).

In some embodiments, the composition comprises biomass from chestnut oak (Quercus prinus).

In some embodiments, the composition comprises biomass from chinkapin oak (Quercus muhlenbergii).

In some embodiments, the composition comprises biomass from canyon live oak (Quercus chrysolepis).

In some embodiments, the composition comprises biomass from overcup oak (Quercus lyrata).

In some embodiments, the composition comprises biomass from English oak (Quercus robur).

In some embodiments, the composition comprises biomass from red oak.

In some embodiments, the composition comprises biomass from northern red oak (Quercus rubra).

In some embodiments, the composition comprises biomass from eastern black oak (Quercus velutina).

In some embodiments, the composition comprises biomass from laurel oak (Quercus laurifolia).

In some embodiments, the composition comprises biomass from southern red oak (Quercus falcata).

In some embodiments, the composition comprises biomass from water oak (Quercus nigra).

In some embodiments, the composition comprises biomass from willow oak (Quercus phellos).

In some embodiments, the composition comprises biomass from Nuttall's oak (Quercus texana).

In some embodiments, the composition comprises biomass from okoumé (Aucoumea klaineana).

In some embodiments, the composition comprises biomass from olive (Olea europaea).

In some embodiments, the composition comprises biomass from pink ivory (Berchemia zeyheri).

In some embodiments, the composition comprises biomass from poplar.

In some embodiments, the composition comprises biomass from balsam poplar (Populus balsamifera).

In some embodiments, the composition comprises biomass from black poplar (Populus nigra).

In some embodiments, the composition comprises biomass from hybrid black poplar (Populus x canadensis).

In some embodiments, the composition comprises biomass from purpleheart (Peltogyne).

In some embodiments, the composition comprises biomass from Queensland maple (Flindersia brayleyana).

In some embodiments, the composition comprises biomass from Queensland walnut (Endiandra palmerstonii).

In some embodiments, the composition comprises biomass from ramin (Gonystylus).

In some embodiments, the composition comprises biomass from redheart, chakté-coc (Erythroxylon mexicanum).

In some embodiments, the composition comprises biomass from sal (Shorea robusta).

In some embodiments, the composition comprises biomass from sweetgum (Liquidambar styraciflua).

In some embodiments, the composition comprises biomass from sandalwood (Santalum).

In some embodiments, the composition comprises biomass from Australian sandalwood (Santalum spicatum).

In some embodiments, the composition comprises biomass from Indian sandalwood (Santalum album).

In some embodiments, the composition comprises biomass from Hawaiian sandalwood (Santalum ellipticum, Santalum freycinetianum, Santalum paniculatum, Santalum haleakalae).

In some embodiments, the composition comprises biomass from Santalum acuminatum.

In some embodiments, the composition comprises biomass from Santalum yasi.

In some embodiments, the composition comprises biomass from Santalum spicatum.

In some embodiments, the composition comprises biomass from sassafras (Sassafras albidum).

In some embodiments, the composition comprises biomass from southern sassafras (Atherosperma moschatum).

In some embodiments, the composition comprises biomass from satiné (Brosimum rubescens).

In some embodiments, the composition comprises biomass from silky oak (Grevillea robusta).

In some embodiments, the composition comprises biomass from silver wattle (Acacia dealbata).

In some embodiments, the composition comprises biomass from sourwood (Oxydendrum arboreum).

In some embodiments, the composition comprises biomass from Spanish-cedar (Cedrela odorata).

In some embodiments, the composition comprises biomass from Spanish elm (Cordia alliodora).

In some embodiments, the composition comprises biomass from tamboti (Spirostachys africana).

In some embodiments, the composition comprises biomass from teak (Tectona grandis).

In some embodiments, the composition comprises biomass from Thailand rosewood (Dalbergia cochinchinensis).

In some embodiments, the composition comprises biomass from tupelo (Nyssa).

In some embodiments, the composition comprises biomass from black tupelo (Nyssa sylvatica).

In some embodiments, the composition comprises biomass from tulip tree (Liriodendron tulipifera).

In some embodiments, the composition comprises biomass from turpentine (Syncarpia glomulifera).

In some embodiments, the composition comprises biomass from walnut (Juglans).

In some embodiments, the composition comprises biomass from Eastern black walnut (Juglans nigra).

In some embodiments, the composition comprises biomass from common walnut (Juglans regia).

In some embodiments, the composition comprises biomass from wenge (Millettia laurentii).

In some embodiments, the composition comprises biomass from panga-panga (Millettia stuhlmannii).

In some embodiments, the composition comprises biomass from willow (Salix).

In some embodiments, the composition comprises biomass from black willow (Salix nigra).

In some embodiments, the composition comprises biomass from cricket-bat willow (Salix alba Caerulea).

In some embodiments, the composition comprises biomass from white willow (Salix alba).

In some embodiments, the composition comprises biomass from weeping willow (Salix babylonica).

In some embodiments, the composition comprises biomass from zingana (Microberlinia brazzavillensis).

In some embodiments, condensing the portion of the vapor to produce a distillate comprises contacting the vapor with a solvent.

In some embodiments, the distillate is dissolved in a solvent.

In some embodiments, the solvent is ethanol.

In some embodiments, the solvent is water.

In some embodiments, the solvent is propylene glycol.

In some embodiments, the solvent is glycerol.

In some embodiments, the solvent is a triglyceride.

In some embodiments, the residual solids and liquids comprise tannins. In some specific embodiments, the residual solids and liquids comprise tannins; and the vapor lacks tannins.

In some embodiments, the composition comprises tannins; and the residual solids and liquids comprise at least 90 percent by mass of the tannins of the composition. In some specific embodiments, the composition comprises tannins; and the residual solids and liquids comprise at least 95 percent by mass of the tannins of the composition. In some very specific embodiments, the composition comprises tannins; and the residual solids and liquids comprise at least 98 percent by mass of the tannins of the composition.

In some embodiments, the residual solids and liquids comprise gallotannins. In some specific embodiments, the residual solids and liquids comprise gallotannins; and the vapor lacks gallotannins.

In some embodiments, the composition comprises gallotannins; and the residual solids and liquids comprise at least 90 percent by mass of the gallotannins of the composition. In some specific embodiments, the composition comprises gallotannins; and the residual solids and liquids comprise at least 95 percent by mass of the gallotannins of the composition. In some very specific embodiments, the composition comprises gallotannins; and the residual solids and liquids comprise at least 98 percent by mass of the gallotannins of the composition.

In some embodiments, the residual solids and liquids comprise ellagitannins. In some specific embodiments, the residual solids and liquids comprise ellagitannins; and the vapor lacks ellagitannins.

In some embodiments, the composition comprises ellagitannins; and the residual solids and liquids comprise at least 90 percent by mass of the ellagitannins of the composition. In some specific embodiments, the composition comprises ellagitannins; and the residual solids and liquids comprise at least 95 percent by mass of the ellagitannins of the composition. In some very specific embodiments, the composition comprises ellagitannins; and the residual solids and liquids comprise at least 98 percent by mass of the ellagitannins of the composition.

In some embodiments, the residual solids and liquids comprise condensed tannins. In some specific embodiments, the residual solids and liquids comprise condensed tannins; and the vapor lacks condensed tannins.

In some embodiments, the composition comprises condensed tannins; and the residual solids and liquids comprise at least 90 percent by mass of the condensed tannins of the composition. In some specific embodiments, the composition comprises condensed tannins; and the residual solids and liquids comprise at least 95 percent by mass of the condensed tannins of the composition. In some very specific embodiments, the composition comprises condensed tannins; and the residual solids and liquids comprise at least 98 percent by mass of the condensed tannins of the composition.

In some embodiments, the residual solids and liquids comprise 1,6-bis-O-galloyl-beta-D-glucose. In some specific embodiments, the residual solids and liquids comprise 1,6-bis-O-galloyl-beta-D-glucose; and the vapor lacks 1,6-bis-O-galloyl-beta-D-glucose.

In some embodiments, the composition comprises 1,6-bis-O-galloyl-beta-D-glucose; and the residual solids and liquids comprise at least 90 percent by mass of the 1,6-bis-O-galloyl-beta-D-glucose of the composition. In some specific embodiments, the composition comprises 1,6-bis-O-galloyl-beta-D-glucose; and the residual solids and liquids comprise at least 95 percent by mass of the 1,6-bis-O-galloyl-beta-D-glucose of the composition. In some very specific embodiments, the composition comprises 1,6-bis-O-galloyl-beta-D-glucose; and the residual solids and liquids comprise at least 98 percent by mass of the 1,6-bis-O-galloyl-beta-D-glucose of the composition.

In some embodiments, the residual solids and liquids comprise 33-O-ethylvescalagin. In some specific embodiments, the residual solids and liquids comprise 33-O-ethylvescalagin; and the vapor lacks 33-O-ethylvescalagin.

In some embodiments, the composition comprises 33-O-ethylvescalagin; and the residual solids and liquids comprise at least 90 percent by mass of the 33-O-ethylvescalagin of the composition. In some specific embodiments, the composition comprises 33-O-ethylvescalagin; and the residual solids and liquids comprise at least 95 percent by mass of the 33-O-ethylvescalagin of the composition. In some very specific embodiments, the composition comprises 33-O-ethylvescalagin; and the residual solids and liquids comprise at least 98 percent by mass of the 33-O-ethylvescalagin of the composition.

In some embodiments, the residual solids and liquids comprise castalagin. In some specific embodiments, the residual solids and liquids comprise castalagin; and the vapor lacks castalagin.

In some embodiments, the composition comprises castalagin; and the residual solids and liquids comprise at least 90 percent by mass of the castalagin of the composition. In some specific embodiments, the composition comprises castalagin; and the residual solids and liquids comprise at least 95 percent by mass of the castalagin of the composition. In some very specific embodiments, the composition comprises castalagin; and the residual solids and liquids comprise at least 98 percent by mass of the castalagin of the composition.

In some embodiments, the residual solids and liquids comprise castalin. In some specific embodiments, the residual solids and liquids comprise castalin; and the vapor lacks castalin.

In some embodiments, the composition comprises castalin; and the residual solids and liquids comprise at least 90 percent by mass of the castalin of the composition. In some specific embodiments, the composition comprises castalin; and the residual solids and liquids comprise at least 95 percent by mass of the castalin of the composition. In some very specific embodiments, the composition comprises castalin; and the residual solids and liquids comprise at least 98 percent by mass of the castalin of the composition.

In some embodiments, the residual solids and liquids comprise casuarinin. In some specific embodiments, the residual solids and liquids comprise casuarinin; and the vapor lacks casuarinin.

In some embodiments, the composition comprises casuarinin; and the residual solids and liquids comprise at least 90 percent by mass of the casuarinin of the composition. In some specific embodiments, the composition comprises casuarinin; and the residual solids and liquids comprise at least 95 percent by mass of the casuarinin of the composition. In some very specific embodiments, the composition comprises casuarinin; and the residual solids and liquids comprise at least 98 percent by mass of the casuarinin of the composition.

In some embodiments, the residual solids and liquids comprise ellagic acid. In some specific embodiments, the residual solids and liquids comprise ellagic acid; and the vapor lacks ellagic acid.

In some embodiments, the composition comprises ellagic acid; and the residual solids and liquids comprise at least 90 percent by mass of the ellagic acid of the composition. In some specific embodiments, the composition comprises ellagic acid; and the residual solids and liquids comprise at least 95 percent by mass of the ellagic acid of the composition. In some very specific embodiments, the composition comprises ellagic acid; and the residual solids and liquids comprise at least 98 percent by mass of the ellagic acid of the composition.

In some embodiments, the residual solids and liquids comprise grandinin. In some specific embodiments, the residual solids and liquids comprise grandinin; and the vapor lacks grandinin.

In some embodiments, the composition comprises grandinin; and the residual solids and liquids comprise at least 90 percent by mass of the grandinin of the composition. In some specific embodiments, the composition comprises grandinin; and the residual solids and liquids comprise at least 95 percent by mass of the grandinin of the composition. In some very specific embodiments, the composition comprises grandinin; and the residual solids and liquids comprise at least 98 percent by mass of the grandinin of the composition.

In some embodiments, the residual solids and liquids comprise pedunculagin. In some specific embodiments, the residual solids and liquids comprise pedunculagin; and the vapor lacks pedunculagin.

In some embodiments, the composition comprises pedunculagin; and the residual solids and liquids comprise at least 90 percent by mass of the pedunculagin of the composition. In some specific embodiments, the composition comprises pedunculagin; and the residual solids and liquids comprise at least 95 percent by mass of the pedunculagin of the composition. In some very specific embodiments, the composition comprises pedunculagin; and the residual solids and liquids comprise at least 98 percent by mass of the pedunculagin of the composition.

In some embodiments, the residual solids and liquids comprise proanthocyanidin A1. In some specific embodiments, the residual solids and liquids comprise proanthocyanidin A1; and the vapor lacks proanthocyanidin A1.

In some embodiments, the composition comprises proanthocyanidin A1; and the residual solids and liquids comprise at least 90 percent by mass of the proanthocyanidin A1 of the composition. In some specific embodiments, the composition comprises proanthocyanidin A1; and the residual solids and liquids comprise at least 95 percent by mass of the proanthocyanidin A1 of the composition. In some very specific embodiments, the composition comprises proanthocyanidin A1; and the residual solids and liquids comprise at least 98 percent by mass of the proanthocyanidin A1 of the composition.

In some embodiments, the residual solids and liquids comprise proanthocyanidin A2. In some specific embodiments, the residual solids and liquids comprise proanthocyanidin A2; and the vapor lacks proanthocyanidin A2.

In some embodiments, the composition comprises proanthocyanidin A2; and the residual solids and liquids comprise at least 90 percent by mass of the proanthocyanidin A2 of the composition. In some specific embodiments, the composition comprises proanthocyanidin A2; and the residual solids and liquids comprise at least 95 percent by mass of the proanthocyanidin A2 of the composition. In some very specific embodiments, the composition comprises proanthocyanidin A2; and the residual solids and liquids comprise at least 98 percent by mass of the proanthocyanidin A2 of the composition.

In some embodiments, the residual solids and liquids comprise proanthocyanidin C1. In some specific embodiments, the residual solids and liquids comprise proanthocyanidin C1; and the vapor lacks proanthocyanidin C1.

In some embodiments, the composition comprises proanthocyanidin C1; and the residual solids and liquids comprise at least 90 percent by mass of the proanthocyanidin C1 of the composition. In some specific embodiments, the composition comprises proanthocyanidin C1; and the residual solids and liquids comprise at least 95 percent by mass of the proanthocyanidin C1 of the composition. In some very specific embodiments, the composition comprises proanthocyanidin C1; and the residual solids and liquids comprise at least 98 percent by mass of the proanthocyanidin C1 of the composition.

In some embodiments, the residual solids and liquids comprise proanthocyanidin C2. In some specific embodiments, the residual solids and liquids comprise proanthocyanidin C2; and the vapor lacks proanthocyanidin C2.

In some embodiments, the composition comprises proanthocyanidin C2; and the residual solids and liquids comprise at least 90 percent by mass of the proanthocyanidin C2 of the composition. In some specific embodiments, the composition comprises proanthocyanidin C2; and the residual solids and liquids comprise at least 95 percent by mass of the proanthocyanidin C2 of the composition. In some very specific embodiments, the composition comprises proanthocyanidin C2; and the residual solids and liquids comprise at least 98 percent by mass of the proanthocyanidin C2 of the composition.

In some embodiments, the residual solids and liquids comprise procyanidin B1. In some specific embodiments, the residual solids and liquids comprise procyanidin B1; and the vapor lacks procyanidin B1.

In some embodiments, the composition comprises procyanidin B1; and the residual solids and liquids comprise at least 90 percent by mass of the procyanidin B1 of the composition. In some specific embodiments, the composition comprises procyanidin B1; and the residual solids and liquids comprise at least 95 percent by mass of the procyanidin B1 of the composition. In some very specific embodiments, the composition comprises procyanidin B1; and the residual solids and liquids comprise at least 98 percent by mass of the procyanidin B1 of the composition.

In some embodiments, the residual solids and liquids comprise procyanidin B2. In some specific embodiments, the residual solids and liquids comprise procyanidin B2; and the vapor lacks procyanidin B2.

In some embodiments, the composition comprises procyanidin B2; and the residual solids and liquids comprise at least 90 percent by mass of the procyanidin B2 of the composition. In some specific embodiments, the composition comprises procyanidin B2; and the residual solids and liquids comprise at least 95 percent by mass of the procyanidin B2 of the composition. In some very specific embodiments, the composition comprises procyanidin B2; and the residual solids and liquids comprise at least 98 percent by mass of the procyanidin B2 of the composition.

In some embodiments, the residual solids and liquids comprise procyanidin B2g. In some specific embodiments, the residual solids and liquids comprise procyanidin B2g; and the vapor lacks procyanidin B2g.

In some embodiments, the composition comprises procyanidin B2g; and the residual solids and liquids comprise at least 90 percent by mass of the procyanidin B2g of the composition. In some specific embodiments, the composition comprises procyanidin B2g; and the residual solids and liquids comprise at least 95 percent by mass of the procyanidin B2g of the composition. In some very specific embodiments, the composition comprises procyanidin B2g; and the residual solids and liquids comprise at least 98 percent by mass of the procyanidin B2g of the composition.

In some embodiments, the residual solids and liquids comprise procyanidin B4. In some specific embodiments, the residual solids and liquids comprise procyanidin B4; and the vapor lacks procyanidin B4.

In some embodiments, the composition comprises procyanidin B4; and the residual solids and liquids comprise at least 90 percent by mass of the procyanidin B4 of the composition. In some specific embodiments, the composition comprises procyanidin B4; and the residual solids and liquids comprise at least 95 percent by mass of the procyanidin B4 of the composition. In some very specific embodiments, the composition comprises procyanidin B4; and the residual solids and liquids comprise at least 98 percent by mass of the procyanidin B4 of the composition.

In some embodiments, the residual solids and liquids comprise procyanidin B7. In some specific embodiments, the residual solids and liquids comprise procyanidin B7; and the vapor lacks procyanidin B7.

In some embodiments, the composition comprises procyanidin B7; and the residual solids and liquids comprise at least 90 percent by mass of the procyanidin B7 of the composition. In some specific embodiments, the composition comprises procyanidin B7; and the residual solids and liquids comprise at least 95 percent by mass of the procyanidin B7 of the composition. In some very specific embodiments, the composition comprises procyanidin B7; and the residual solids and liquids comprise at least 98 percent by mass of the procyanidin B7 of the composition.

In some embodiments, the residual solids and liquids comprise roburin A. In some specific embodiments, the residual solids and liquids comprise roburin A; and the vapor lacks roburin A.

In some embodiments, the composition comprises roburin A; and the residual solids and liquids comprise at least 90 percent by mass of the roburin A of the composition. In some specific embodiments, the composition comprises roburin A; and the residual solids and liquids comprise at least 95 percent by mass of the roburin A of the composition. In some very specific embodiments, the composition comprises roburin A; and the residual solids and liquids comprise at least 98 percent by mass of the roburin A of the composition.

In some embodiments, the residual solids and liquids comprise roburin B. In some specific embodiments, the residual solids and liquids comprise roburin B; and the vapor lacks roburin B.

In some embodiments, the composition comprises roburin B; and the residual solids and liquids comprise at least 90 percent by mass of the roburin B of the composition. In some specific embodiments, the composition comprises roburin B; and the residual solids and liquids comprise at least 95 percent by mass of the roburin B of the composition. In some very specific embodiments, the composition comprises roburin B; and the residual solids and liquids comprise at least 98 percent by mass of the roburin B of the composition.

In some embodiments, the residual solids and liquids comprise roburin C. In some specific embodiments, the residual solids and liquids comprise roburin C; and the vapor lacks roburin C.

In some embodiments, the composition comprises roburin C; and the residual solids and liquids comprise at least 90 percent by mass of the roburin C of the composition. In some specific embodiments, the composition comprises roburin C; and the residual solids and liquids comprise at least 95 percent by mass of the roburin C of the composition. In some very specific embodiments, the composition comprises roburin C; and the residual solids and liquids comprise at least 98 percent by mass of the roburin C of the composition.

In some embodiments, the residual solids and liquids comprise roburin D. In some specific embodiments, the residual solids and liquids comprise roburin D; and the vapor lacks roburin D.

In some embodiments, the composition comprises roburin D; and the residual solids and liquids comprise at least 90 percent by mass of the roburin D of the composition. In some specific embodiments, the composition comprises roburin D; and the residual solids and liquids comprise at least 95 percent by mass of the roburin D of the composition. In some very specific embodiments, the composition comprises roburin D; and the residual solids and liquids comprise at least 98 percent by mass of the roburin D of the composition.

In some embodiments, the residual solids and liquids comprise roburin E. In some specific embodiments, the residual solids and liquids comprise roburin E; and the vapor lacks roburin E.

In some embodiments, the composition comprises roburin E; and the residual solids and liquids comprise at least 90 percent by mass of the roburin E of the composition. In some specific embodiments, the composition comprises roburin E; and the residual solids and liquids comprise at least 95 percent by mass of the roburin E of the composition. In some very specific embodiments, the composition comprises roburin E; and the residual solids and liquids comprise at least 98 percent by mass of the roburin E of the composition.

In some embodiments, the residual solids and liquids comprise sanguisorbic acid. In some specific embodiments, the residual solids and liquids comprise sanguisorbic acid; and the vapor lacks sanguisorbic acid.

In some embodiments, the composition comprises sanguisorbic acid; and the residual solids and liquids comprise at least 90 percent by mass of the sanguisorbic acid of the composition. In some specific embodiments, the composition comprises sanguisorbic acid; and the residual solids and liquids comprise at least 95 percent by mass of the sanguisorbic acid of the composition. In some very specific embodiments, the composition comprises sanguisorbic acid; and the residual solids and liquids comprise at least 98 percent by mass of the sanguisorbic acid of the composition.

In some embodiments, the residual solids and liquids comprise tannic acid. In some specific embodiments, the residual solids and liquids comprise tannic acid; and the vapor lacks tannic acid.

In some embodiments, the composition comprises tannic acid; and the residual solids and liquids comprise at least 90 percent by mass of the tannic acid of the composition. In some specific embodiments, the composition comprises tannic acid; and the residual solids and liquids comprise at least 95 percent by mass of the tannic acid of the composition. In some very specific embodiments, the composition comprises tannic acid; and the residual solids and liquids comprise at least 98 percent by mass of the tannic acid of the composition.

In some embodiments, the residual solids and liquids comprise valoneic acid dilactone. In some specific embodiments, the residual solids and liquids comprise valoneic acid dilactone; and the vapor lacks valoneic acid dilactone.

In some embodiments, the composition comprises valoneic acid dilactone; and the residual solids and liquids comprise at least 90 percent by mass of the valoneic acid dilactone of the composition. In some specific embodiments, the composition comprises valoneic acid dilactone; and the residual solids and liquids comprise at least 95 percent by mass of the valoneic acid dilactone of the composition. In some very specific embodiments, the composition comprises valoneic acid dilactone; and the residual solids and liquids comprise at least 98 percent by mass of the valoneic acid dilactone of the composition.

In some embodiments, the residual solids and liquids comprise vescalagin. In some specific embodiments, the residual solids and liquids comprise vescalagin; and the vapor lacks vescalagin.

In some embodiments, the composition comprises vescalagin; and the residual solids and liquids comprise at least 90 percent by mass of the vescalagin of the composition. In some specific embodiments, the composition comprises vescalagin; and the residual solids and liquids comprise at least 95 percent by mass of the vescalagin of the composition. In some very specific embodiments, the composition comprises vescalagin; and the residual solids and liquids comprise at least 98 percent by mass of the vescalagin of the composition.

In some embodiments, separating the vapor from the majority of the residual solids and liquids, comprises separating the vapor from at least 90 percent of the residual solids and liquids. In some specific embodiments, separating the vapor from the majority of the residual solids and liquids, comprises separating the vapor from at least 95 percent of the residual solids and liquids.

In some embodiments, condensing the portion of the vapor comprises condensing at least 80 percent of the vapor. In some specific embodiments, condensing the portion of the vapor comprises condensing at least 90 percent of the vapor. In some very specific embodiments, condensing the portion of the vapor comprises condensing at least 98 percent of the vapor.

Various aspects of this disclosure relate to a distillate produced according to a method described anywhere in the disclosure.

Various aspects of this disclosure relate to a product manufactured from a distillate produced according to a method described anywhere in the disclosure.

In some embodiments, the product is an oak extract.

In some embodiments, the product is a wood extract.

In some embodiments, the product is a flavoring.

In some embodiments, the product is a beverage flavoring.

In some embodiments, the product is a food flavoring.

In some embodiments, the product is a functional ingredient.

In some embodiments, the product is an alcoholic beverage flavoring.

In some embodiments, the product is a nonalcoholic beverage flavoring.

In some embodiments, the product is an aromatic cocktail garnish.

In some embodiments, the product is an aromatic bitters.

In some embodiments, the product is an edible extract that is generally regarded as safe by the US Food and Drug Administration (GRAS).

In some embodiments, the product is a flavor that is generally regarded as safe by the US Food and Drug Administration (GRAS).

In some embodiments, the product is an Alcohol and Tobacco Tax and Trade Bureau approved flavor.

In some embodiments, the product is an Alcohol and Tobacco Tax and Trade Bureau approved extract.

In some embodiments, the product is a beverage.

In some embodiments, the product is an alcoholic beverage. In some specific embodiments, the product is a liquor, wine, beer, or cocktail. In some specific embodiments, the product is a nonalcoholic liquor, wine, beer, or cocktail that contains less than 0.5% alcohol by volume. In some embodiments, the product is a beer. In some specific embodiments, the product is a seltzer. In some specific embodiments, the product is a cider. In some specific embodiments, the product is a nonalcoholic beer. In some specific embodiments, the product is a nonalcoholic seltzer.

In some embodiments, the product is a wine. In some specific embodiments, the product is a white wine. In some specific embodiments, the product is a red wine. In some specific embodiments, the product is a nonalcoholic wine that contains less than 0.5% alcohol by volume.

In some embodiments, the product is a liquor. In some specific embodiments, the product is a whiskey. In some specific embodiments, the product is a rum. In some specific embodiments, the product is a brandy. In some specific embodiments, the product is a cognac. In some specific embodiments, the product is a mescal or tequila. In some specific embodiments, the product is a nonalcoholic spirit that contains less than 0.5% alcohol by volume.

In some embodiments, the product is a consumer packaged good.

In some embodiments, the product is a flavoring.

In some embodiments, the product is synthetic vanillin.

In some embodiments, the product is a food sauce.

In some embodiments, the product is a food.

In some embodiments, the product is a dietary supplement.

In some embodiments, the product is a fragrance.

In some embodiments, the product is a scented skin care product.

In some embodiments, the product is a perfume.

In some embodiments, the product is an air freshener.

In some embodiments, the product is a cleaning preparation.

In some embodiments, the product is a soap or detergent.

In some embodiments, the product is a scented candle.

Various aspects of this disclosure relate to an essential oil of a composition, wherein the essential oil is produced from a composition according to a method disclosed anywhere in this disclosure.

### Exemplification. Production of Various Flavor Molecules From Oak

A system for extracting an oil from plant material as described in European Patent No. 3,283,606 was used to extract sawdust obtained from oak obtained from whiskey barrels. The oak was extracted at about 1 kilogram oak into 1 liter ethanol. Extraction conditions were optimized to drive thermal decomposition of the oak to produce various flavor molecules. This allowed increased furfural production from 40 parts per million up to 0.15 percent; guaiacol from 40 parts per billion to 2,500 parts per billion; 2-methoxy-4-methylphenol from 20 parts per billion to 1,600 parts per billion; eugenol from 60 parts per billion to 830 parts per billion; and vanillin from 3.5 parts per million to 22.1 parts per million.

1. A method to manufacture furfural, comprising:
providing a composition comprising a pentose;
transferring energy to the composition to convert the composition into a vapor and residual solids and liquids, wherein converting the composition into the vapor and the residual solids and liquids comprises converting a portion of the pentose into furfural vapor and wherein the vapor comprises the furfural vapor;
separating the vapor from a majority of the residual solids and liquids; and
condensing a portion of the vapor to produce a distillate that comprises furfural.

2-6. (canceled)

7. The method of claim 1, wherein the pentose is a ketone.

8. The method of claim 1, wherein the pentose is xylulose.

9. A method to manufacture guaiacol, comprising providing a composition comprising phenolic molecules selected from monolignols, lignans, and lignin; transferring energy to the composition to convert the composition into a vapor and residual solids and liquids, wherein converting the composition into the vapor and the residual solids and liquids comprises converting a portion of the phenolic molecules into guaiacol vapor and wherein the vapor comprises the guaiacol vapor; separating the vapor from a majority of the residual solids and liquids; and condensing a portion of the vapor to produce a distillate that comprises guaiacol.

10-19. (canceled)

20. A method to manufacture 2,6-dimethoxy-4-(prop-2-enyl)phenol, comprising providing a composition comprising phenolic molecules selected from monolignols, lignans, and lignin; transferring energy to the composition to convert the composition into a vapor and residual solids and liquids, wherein converting the composition into the vapor and the residual solids and liquids comprises converting a portion of the phenolic molecules into 2,6-dimethoxy-4-(prop-2-enyl)phenol vapor and wherein the vapor comprises the 2,6-dimethoxy-4-(prop-2-enyl)phenol vapor; separating the vapor from a majority of the residual solids and liquids; and condensing a portion of the vapor to produce a distillate that comprises 2,6-dimethoxy-4-(prop-2-enyl)phenol.

21-68. (canceled)

69. The method of claim 1, comprising bombarding the composition with at least 1 liter of a sweep gas per gram of the composition per second.

70-97. (canceled)

98. The method of claim 69, wherein:
the composition is in fluid communication with a gas phase that has a temperature and a pressure;
the composition off-gasses the furfural vapor into the gas phase at a vaporization rate;
the furfural vapor recondenses from the gas phase onto the composition at a condensation rate;
the method comprises converting furfural of the composition into the furfural vapor at a mass transfer rate, which is equal to the vaporization rate minus the condensation rate;
the furfural has a vapor pressure;
increasing the vapor pressure of the furfural increases the mass transfer rate; and
the bombarding both increases the vapor pressure of the furfural and increases the mass transfer rate.

99-104. (canceled)

105. The method of claim 69, wherein the bombarding suspends at least 75 percent of the composition in the gas phase.

106-117. (canceled)

118. The method of claim 69, wherein the bombarding performs work that separates the vaporized molecule from the majority of the residual solids and liquids.

119-142. (canceled)

143. The method of claim 69, wherein the sweep gas has a Reynolds number of at least 1 during the bombarding.

144-152. (canceled)

153. The method of claim 1, wherein the composition has a surface-area-to-volume ratio that is greater than 500 per meter.

154-181. (canceled)

182. The method of claim 1, wherein:
the composition comprises biological cells;
the biological cells have cell walls; and
the method comprises generating sufficient pressure within the biological cells to rupture at least 10 percent of the cell walls.

183-214. (canceled)

215. The method of claim 47, wherein:
the composition comprises a starting concentration of furfural by mass;
the distillate comprises an ending concentration of furfural by mass; and
the ending concentration is at least 5 times greater than the starting concentration.

216-237. (canceled)

238. The method of claim 1, wherein the composition comprises sawdust.

239-502. (canceled)

503. The method of claim 1, wherein the composition comprises biomass from oak (Quercus).

504-596. (canceled)

597. The method of claim 1, wherein the residual solids and liquids comprise 1,6-bis-O-galloyl-beta-D-glucose.

598-726. (canceled)

727. A distillate produced according to the method of claim 1.

728. A product manufactured from a distillate produced according to the method of claim 1.

729. The product of claim 728, wherein the product is a beverage.

730-748. (canceled)

749. An essential oil, wherein the essential oil is produced according to the method of claim 1.
